SEQUENCE LISTING (38 sequences)

SEQ ID NO: 1 - protein, 113 aa, Artificial Sequence
  IL-13 Superkine
PGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 2 - protein, 113 aa, Artificial Sequence
  Modified IL-13 Superkine
PGPVPPSTAVRALIEELINITQNQKAPLCNGSMVWSINRTAGMYCAALESLINVSGCSAIEKTQDMLSGFCPHKVSAGQFSSLHVRSSKIEVAQFVKDLLFHLRTLFREGQFN

SEQ ID NO: 3 - protein, 113 aa, Artificial Sequence
  Modified IL-13 Superkine
PGPVPPSTAIRELIEELINITQNQKAPLCNGSMVWSINLTAGMYCAALESLINVSGCSAIEKTQRMLSGFCPHKVSAGQFSSLHVRGSKIEVAQFVKDLLHHLRALFREGQFN

SEQ ID NO: 4 - protein, 113 aa, Artificial Sequence
  Modified IL-13 Superkine
PGPVPPSTAVRELIEELINITQNQKAPLCNGSMVWSINRTAGMYCAALESLINVSGCSAIEKTQRMLSGFCPHKVSAGQFSSLHVRSSKIEVAQFVKDLLFHLRTLFREGQFN

SEQ ID NO: 5 - protein, 113 aa, Artificial Sequence
  Modified IL-13 Superkine
PGPVPPSTALIELIEELINITQNQKAPLCNGSMVWSINLTAGIYCAALESLINVSGCSAIEKTQRMLSGFCPHKVSAGQFSSLHVKGSKIEVAQFVKDLLHHLRALMREGQFN

SEQ ID NO: 6 - protein, 113 aa, Artificial Sequence
  Modified IL-13 Superkine
PGPVPPSTAIRELIEELLNITQNQKAPLCNGSMVWSINLTAGMYCAALESLINVSGCSAIEKTQRMLSGFCPHKVSAGQFSSLHVMKSKIEVAQFVKDLLHHLRALFREGQFN

SEQ ID NO: 7 - protein, 113 aa, Artificial Sequence
  Modified IL-13 Superkine
PGPVPPSTAIRELIEELINITQNQKAPLCNGSMVWSINLTAGMYCAALESLINVSGCSAIEKTQRMLSGFCPHKVSAGQFSSLHVRSSRIEVAQFVKDLLHHLRTLFREGQFN

SEQ ID NO: 8 - protein, 113 aa, Artificial Sequence
  Modified IL-13 Superkine
PGPVPPSTALRELIEELINITQNEKAPLCNGSMVWSINLTAGIYCAALESLINVSGCSAIEKTQRMLSGFCPHKVSAGQFSSLHVTGSKIEVAQFVKDLLYHLRALFREGQFN

SEQ ID NO: 9 - protein, 113 aa, Artificial Sequence
  Modified IL-13 Superkine
PGPVPPSTALSELIEELINITQNQKAPLCNGSMVWSINPTAGMYCAALESLINVSGCSAIEKTQRMLSGFCPHKVAAGQFSSLHDKGSMIEVAQFVKDLLYHLRTLFREGQFN

SEQ ID NO: 10 - protein, 113 aa, Artificial Sequence
  Modified IL-13 Superkine
PGPVPPSTATRELIEELINITQNQKAPLCNGSMVWSINLTADMYCAALESLINVSGCSAIEKTQRMLSGFCPHKVSVGQFSSLHVRGSKIEVAQFVKDLLYHLRTLFREGQFN

SEQ ID NO: 11 - protein, 113 aa, Artificial Sequence
  Modified IL-13 Superkine
PGPVPPSTADIELIAELINITQNQKAPLCNGSMVWSINLTADMYCAALESLINVSGCSAIEKTQRMLSGFCPHKVSAGQFSSLHVKKTRIEVAQFVKDLLLHLKKLFKEGQFN

SEQ ID NO: 12 - protein, 113 aa, Artificial Sequence
  Modified IL-13 Superkine
PGPVPPSTAARELIEELVNITQNQKAPLCNGSMVWSINLTAGMYCAALESLINVSGCSAIEKTQRMLSGFCPHKVSAGQLSSLHVTGKRIEVAQFVKDLLNHLRALFKEGQFN

SEQ ID NO: 13 - protein, 113 aa, Artificial Sequence
  Modified IL-13 Superkine
PGPVPPSTAVRELIEELVNITQNQKAPLCNGSMVWSINLTAGMYCAALESLINVSGCSAIEKTQRMLSGFCPHKVSAGQFSSLHVRDTRIEVAQFVKDLLNHLKELFTEGQFN

SEQ ID NO: 14 - protein, 113 aa, Artificial Sequence
  Modified IL-13 Superkine
PGPVPPSTALSELMEELVNITQNQKAPLCNGSMVWSINLTAGMYCAALESLINVSGCSAIEKTQRMLSGFCPHKVSAGQFSSLHVRDSKIEVAQFVKDLLNHLKALFKEGQFN

SEQ ID NO: 15 - protein, 112 aa, Artificial Sequence
  Modified IL-13 Superkine
GPVPPSTAFRELIEELVNITQNQKAPLCNGSMVWSINLTAGMYCAALESLINVSGCSAIEKTQRMLSGFCPHKVSPGQFSSLHVTNSRIEVAQFVKDLLNHLKALFKEGQYN

SEQ ID NO: 16 - protein, 112 aa, Artificial Sequence
  Modified IL-13 Superkine
GPVPPSTAHLELIEELINITQNQKAPLCNGSMVWSINLTAGMYCAALESLINVSGCSAIEKTQRMLSGFCPHKVSAGQFSSLHVKETRIEVAQFVKDLLNHLKTLFKEGQFN

SEQ ID NO: 17 - protein, 113 aa, Artificial Sequence
  Modified IL-13 Superkine
PGPVPPSTAHLELIEELINITQNQKAPLCNGSMVWSINPTAGMYCAALESLINVSGCSAIEKTQRMLSGFCPHKVSAGQFSSLHVMDTRIEVAQFVKDLLLHLKKLFKEGQFN

SEQ ID NO: 18 - protein, 113 aa, Artificial Sequence
  Modified IL-13 Superkine
PGPVPPSTAHRELIEELVNITQNQKAPLCNGSMVWSINLTAGMYCAALESLINVSGCSAIEKTQRMLSGFCPHKVSAGQFSSLHVTGRKIEVAQFVKDLLLHLKKLFKEGQFN

SEQ ID NO: 19 - protein, 113 aa, Artificial Sequence
  Modified IL-13 Superkine
PGPVPPSTAHRELIEELVNITQNQKAPLCNGSMVWRINRTAGMYCAALESLINVSGCSAIEKTQRMLSGFCPHKVSAGQFSSLHVMDSRIEVAQFVKDLLNHLRALFKEGQFN

SEQ ID NO: 20 - protein, 113 aa, Artificial Sequence
  Modified IL-13 Superkine
PGPVPPSTAARELIEELFNITQNQKAPLCNGSMVWSINLTAGMYCAALESLINVSGCSAIEKTKRMLSGFCPHKVSAGQFPSLHVKKTRIEVAQFVKDLLIHLRKLFKEGQFN

SEQ ID NO: 21 - protein, 113 aa, Artificial Sequence
  Modified IL-13 Superkine
PGPVPPSTALIELIEELINITQNQKAPLCNGSMVWSINLTAGMYCAALESLINVSGCSAIEKTQRMLSGFCPHKVSAGQFSSLHVKGSKIEVAQFVKDLLHHLRALMREGQFN

SEQ ID NO: 22 - protein, 113 aa, Artificial Sequence; identical to SEQ ID NO: 6
  Modified IL-13 Superkine

SEQ ID NO: 23 - protein, 113 aa, Artificial Sequence; identical to SEQ ID NO: 3
  Modified IL-13 Superkine

SEQ ID NO: 24 - protein, 113 aa, Artificial Sequence; identical to SEQ ID NO: 7
  Modified IL-13 Superkine

SEQ ID NO: 25 - protein, 113 aa, Artificial Sequence
  Modified IL-13 Superkine
PGPVPPSTAVRELIEELINITQNQKAPLCNGSMVWSINLTAGMYCAALESLINVSGCSAIEKTQRMLSGFCPHKVSAGQFSSLHVRSSKIEVAQFVKDLLFHLRTLFREGQFN

SEQ ID NO: 26 - protein, 113 aa, Artificial Sequence
  Modified IL-13 Superkine
PGPVPPSTALRELIEELINITQNQKAPLCNGSMVWSINLTAGMYCAALESLINVSGCSAIEKTQRMLSGFCPHKVSAGQFSSLHVTGSKIEVAQFVKDLLYHLRALFREGQFN

SEQ ID NO: 27 - protein, 113 aa, Artificial Sequence
  Modified IL-13 Superkine
PGPVPPSTALSELIEELINITQNQKAPLCNGSMVWSINLTAGMYCAALESLINVSGCSAIEKTQRMLSGFCPHKVSAGQFSSLHVKGSMIEVAQFVKDLLYHLRTLFREGQFN

SEQ ID NO: 28 - protein, 113 aa, Artificial Sequence
  Modified IL-13 Superkine
PGPVPPSTATRELIEELINITQNQKAPLCNGSMVWSINLTAGMYCAALESLINVSGCSAIEKTQRMLSGFCPHKVSAGQFSSLHVRGSKIEVAQFVKDLLYHLRTLFREGQFN

SEQ ID NO: 29 - protein, 113 aa, Artificial Sequence
  Modified IL-13 Superkine
PGPVPPSTADIELIEELINITQNQKAPLCNGSMVWSINLTAGMYCAALESLINVSGCSAIEKTQRMLSGFCPHKVSAGQFSSLHVKKTRIEVAQFVKDLLLHLKKLFKEGQFN

SEQ ID NO: 30 - protein, 113 aa, Artificial Sequence
  Modified IL-13 Superkine
PGPVPPSTAARELIEELVNITQNQKAPLCNGSMVWSINLTAGMYCAALESLINVSGCSAIEKTQRMLSGFCPHKVSAGQFSSLHVTGKRIEVAQFVKDLLNHLRALFKEGQFN

SEQ ID NO: 31 - protein, 113 aa, Artificial Sequence; identical to SEQ ID NO: 13
  Modified IL-13 Superkine

SEQ ID NO: 32 - protein, 113 aa, Artificial Sequence; identical to SEQ ID NO: 14
  Modified IL-13 Superkine

SEQ ID NO: 33 - protein, 113 aa, Artificial Sequence
  Modified IL-13 Superkine
PGPVPPSTAHLELIEELINITQNQKAPLCNGSMVWSINLTAGMYCAALESLINVSGCSAIEKTQRMLSGFCPHKVSAGQFSSLHVKETRIEVAQFVKDLLNHLKTLFKEGQFN

SEQ ID NO: 34 - protein, 113 aa, Artificial Sequence
  Modified IL-13 Superkine
PGPVPPSTAHLELIEELINITQNQKAPLCNGSMVWSINLTAGMYCAALESLINVSGCSAIEKTQRMLSGFCPHKVSAGQFSSLHVMDTRIEVAQFVKDLLLHLKKLFKEGQFN

SEQ ID NO: 35 - protein, 113 aa, Artificial Sequence; identical to SEQ ID NO: 18
  Modified IL-13 Superkine

SEQ ID NO: 36 - protein, 113 aa, Artificial Sequence
  Modified IL-13 Superkine
PGPVPPSTAHRELIEELVNITQNQKAPLCNGSMVWSINLTAGMYCAALESLINVSGCSAIEKTQRMLSGFCPHKVSAGQFSSLHVMDSRIEVAQFVKDLLNHLRALFKEGQFN

SEQ ID NO: 37 - protein, 113 aa, Artificial Sequence
  Modified IL-13 Superkine
PGPVPPSTAARELIEELFNITQNQKAPLCNGSMVWSINLTAGMYCAALESLINVSGCSAIEKTQRMLSGFCPHKVSAGQFSSLHVKKTRIEVAQFVKDLLIHLRKLFKEGQFN

SEQ ID NO: 38 - protein, 113 aa, Artificial Sequence
  Modified IL-13 Superkine
PGPVPPSTAVRALIEELINITQNQKAPLCNGSMVWSINLTAGMYCAALESLINVSGCSAIEKTQDMLSGFCPHKVSAGQFSSLHVRSSKIEVAQFVKDLLFHLRTLFREGQFN

(19) United States
(12) Patent Application Publication
(10) Pub. No.: US 2020/0095301 A1
(43) Pub. Date: Mar. 26, 2020

(54) IL-13 SUPERKINE: IMMUNE CELL TARGETING CONSTRUCTS AND METHODS OF USE THEREOF

(71) Applicants: The Board of Trustees of the Leland Stanford Junior University, Stanford, CA (US); Medicenna Therapeutics Inc., Toronto (CA)

(72) Inventors: Kenan Christopher Garcia, Menlo Park, CA (US); Ignacio Moraga Gonzalez, Palo Alto, CA (US); Fahar Merchant, Vancouver (CA)

(21) Appl. No.: 16/470,098

(22) PCT Filed: Dec. 14, 2017
(86) PCT No.: PCT/US17/66529

§ 371 Date: Jun. 14, 2019

Related U.S. Application Data
(60) Provisional application No. 62/434,171, filed on Dec. 14, 2016.

(51) Int. Cl. C07K 14/54; C07K 14/725; A61K 35/17; C07K 14/705; C12N 5/0783; C12N 5/0784; C07K 16/28

(52) U.S. Cl. CPC C07K 14/5437; C07K 14/7051; A61K 35/17; C07K 14/70521; A61K 38/00; C12N 5/0639; C12N 5/0646; C07K 16/2809; C12N 5/0636

(57) ABSTRACT

Methods and compositions are provided for enhancing anti-tumor effector immune cells with a targeting construct a human IL-13 superkine. Cytokine or additional co-stimulatory sequences may also be included to enhance the tumoricidal effects of the cells.

### CROSS REFERENCE TO RELATED APPLICATION

This application claims the benefit of U.S. Provisional Application No. 62/434,171, filed on Dec. 14, 2016, all of which is expressly incorporated herein by reference in its entirety.

### BACKGROUND

Interleukin-13 (IL-13) is a cytokine secreted by T lymphocytes and mast cells, which shares several biological activities with IL-4, as a mediator of allergic inflammation and disease. IL-13 is involved in the allergic response via its actions on epithelial and smooth muscle cells. IL-13 induces many features of allergic lung disease, including airway hyperresponsiveness, goblet cell metaplasia and mucus hypersecretion, which all contribute to airway obstruction. IL-13 also induces secretion of chemokines that are required for recruitment of allergic effector cells to the lung.

An important factor in IL-13 biology is the nature of its receptor interactions. Its diverse functions are mediated by a complex receptor system including IL-4 receptor α (IL-4Rα; CD124) and two other cognate cell surface proteins, IL-13Rα1 (CD213a1) and IL-13Rα2 (CD213a2). IL-13Rα1 forms a heterodimer with IL-4Rα that is a signaling IL-13 receptor. In contrast, IL-13Rα2 has been thought to be a decoy receptor due to its short cytoplasmic tail. IL-13Rα2 exists on the cell membrane, intracellularly, and in soluble form. IL-13Rα2 has an extremely high affinity for IL-13, and can out-compete antibodies for IL-13 binding. The other receptor, IL-13Rα1, has a much lower affinity, but is associated with signaling events mediated by IL-4Rα. It induces its effects through a multi-subunit receptor that includes the alpha chain of the IL-4 receptor (IL-4Rα) and IL-13Rα1. Most of the biological effects of IL-13, like those of IL-4, are linked to a single transcription factor, signal transducer and activator of transcription 6 (STAT6).

IL13Rα2 is highly expressed in many tumor types, such as colorectal, glioblastoma, ovarian, head and neck, breast, pancreatic, kidney, and mesothelioma, but not by most normal cells such as immune cells or endothelial cells. IL13Rα2 is also associated with poor prognosis in human cancers and a target for cancer therapy. High IL13Rα2 expression levels have been shown to promote invasion and metastasis of brain, pancreatic, ovarian, breast and colorectal cancers. Increased IL13Rα2 levels were also associated with poor metastasis-free survival of patients with breast cancer. IL13Rα2 expression is also a prognostic marker for glioma malignancy grade and for poor patient survival.

Targeted immunotherapy has emerged as promising field of research in the treatment of malignancies and has received a great deal of interest in recent years. Indeed, cures have been reported of lymphoma patients with engineered or genetically modified T cells targeting CD19 malignant cells. This has increased the focus towards antigens present on cancer cells as targets for gene- and immunotherapy.

Genetic manipulation of autologous or allogeneic T cells or NK cells to specifically target a particular tumor antigen provides a strategy to bypass the failure of cytotoxic immune response induction by most tumor cells. These technologies are based on the genetic modification of human immune cells, where the cells may be extracted from a patient or donor by leukapheresis. Specific cells, usually T-cells, are purified and engineered to express a receptor targeting a cancer antigen of interest. Engineering may utilize transduction by retroviral, lentiviral, transposon, mRNA electroporation, and the like. The immune cells may be expanded to the desired dose, and introduced into a patient. The engineered cells can specifically kill cancer cells through cell-mediated toxicity (cytotoxic T-cells) and/or eliciting an immune response to the cancer cell by immune recognition of tumor, cytokine release and immune cell recruitment.

For example, the application of chimeric antigen receptors (CAR) for immunogene therapy of malignant tumors is a promising strategy in which an antibody or ligand binding domain is fused with the zeta signaling chain of the T cell receptor. The resulting CAR immune cells are redirected by the neospecificity to attack tumors expressing the surface antigen or receptors recognized by the gene-modified T cell receptors and provide cellular therapy that attacks the tumor through normal host immune response in a highly regulated fashion. These cells are free to circulate throughout the brain and systemic circulation, making the need for colocalization and bioavailability less of a problem.

A number of generations of CAR immune cells have been developed. CARs are created by the fusion of a tumour-specific scFv antibody or other extracellular ligand binding domain to either the TCR-associated CD3ζ signalling domain or another intracellular signalling domains from co-stimulatory protein receptors. This structure allows CARs to have the tumor specificity of the B cell antigen receptor, and to activate T cells through the T cell antigen receptor independently of MHC binding. The first-generation CAR contained one intracellular signalling domain, typically with the CD3ζ signalling domain to allow for TCR signalling. Second-generation CARs have two intracellular signalling domains: a co-stimulatory domain comprising either a CD28 or a 4-1BB signalling domain, coupled with a CD3ζ signalling domain. This arrangement enables T-cell activation and proliferation upon antigen recognition by the scFv region of the CAR. The third-generation CARs have two co-stimulatory domains and a CD3ζ signalling domain. The first co-stimulatory domain is either a CD28 or a 4-1BB domain, with the second co-stimulatory domain consisting of either a CD28, a 4-1BB or a OX40 domain. Fourth-generation “armoured CAR T cells” combine a second-generation CAR with the addition of various genes, including cytokine and co-stimulatory ligands, to enhance the tumoricidal effect of the CAR T cells. See, for example, Batlevi et al. (2016) Nature Reviews Clinical Oncology 13:25-40. See also, U.S. Pat. No. 7,741,465 and International Patent Publication No. WO2014127261; all of which are incorporated by reference herein in their entireties.

Alternative approaches to T cell targeting include T cell antigen couplers, as described in International application WO2015/117229, entitled “Trifunctional T cell antigen Coupler and Methods and Uses thereof”, herein specifically incorporated by reference. The T cell antigen coupler system comprises three linked domains: a target-specific polypeptide ligand; a ligand that binds a protein associated with the TCR complex, for example an scFv binding to CD3 (TCR, T-cell receptor) to stimulate T cell activation; and a T cell receptor signaling domain, for example a CD4 transmembrane and intracellular domain to amplify T cell activation. By stimulating T cell activation through the TCR, TACs were engineered to work with the T cell's essential molecular machinery.

Antibody coupled T cell receptors are another approach to T cell targeting. ACTRs are a hybrid approach to CARs and the established monoclonal antibody oncology therapeutics. ACTRs are composed of a typical CAR construct that can bind the heavy chain of an antibody through a high-affinity variant of the Fc receptor CD16. ACTR-T cells can target tumours by binding a ligand targeted to a specific cancer antigen. T cell activation is performed by the CAR module.

Bispecific T cell exchangers (BiTEs) are bispecific antibodies that can bind the TCR of T cells and target tumour cells through two modules: a cancer targeting ligand; and a CD3-binding scFv domain that bridges T cells to the tumor.

Targeted therapies have been developed against IL13Rα2, including bacterial toxins conjugated to IL13, nanoparticles, oncolytic virus, as well as immunotherapies using monoclonal antibodies, IL13Rα2-pulsed dendritic cells, and IL13Rα2-targeted chimeric antigen receptors (see Kahlon et al. (2004) Cancer Research. 64(24):9160-9166; Kong et al. (2012) Clinical Cancer Research. 18(21):5949-5960; Thaci et al. (2014) Neuro-Oncology; and clinical trials NCT02208362, NCT00730613 and NCT01082926).

Biologicals that provide for selective alteration of IL-13 activity are of interest for a number of therapeutic purposes, including the treatment of certain cancers with by engineering of T cell specificities. The present invention addresses this issue.

### SUMMARY

Methods and compositions are provided for enhancing anti-tumor immune effector cells, e.g. T cells, NK cells, etc. with targeted compositions, including without limitation chimeric antigen receptors (CARs); T cell antigen couplers (TACs); antibody coupled T cell receptors (ACTRs); and bispecific T cell exchangers (BiTEs), where an IL-13 superkine provides the target-specific ligand.

The IL-13 superkine sequence is engineered to have: (a) increased affinity for IL-13Rα2, relative to the native human IL-13 protein; and (b) decreased affinity for IL-13Rα1 relative to the native human IL-13 protein. The increase in affinity for human IL-13Rα2 may be at least two-fold, at least 5-fold, at least 10-fold or more relative to the native protein. The decrease in affinity for human IL-13Rα1 may be at least 10-fold, at least 20-fold, at least 50-fold, at least 100-fold, or more.

Amino acid modifications may be made at one or more of the amino acids within the set of contact residues that interact with IL-13Rα1 and IL-13Rα2, which residues include, without limitation, L10, R11, I14, V18, R86, D87, T88, K89, L101, K104, K105 and R108 (for reference purposes the sequence of wild-type human IL-13 is provided herein as SEQ ID NO:1, to which the numbering of amino acids will refer). In other embodiments, modified residues are at two or more, three or more, four or more, five or more, and not more than 14 amino acids within the combined set of contact residues defined above.

The IL-13 interface that contacts IL-13Rα1 and IL-13Rα2 is the same, and thus there can be overlap in the altered residues that control affinity for these two receptors. In some embodiments one or more of the native amino acid residues L10, R11, I14, V18, R86, D87, T88, K89, L101, K104, K105, F107, and R108 is substituted, and provides for an altered affinity for one or both of IL-13Rα1 and IL-13Rα2.

In some embodiments an IL-13 superkine sequence comprises one or more of the amino acids substitutions relative to SEQ ID NO:1: (1) L10F; L10I; L10V; L10A; L10D; L10T; L10H; (2) R11S; R11N; R11H; R11L; R11I; (3) I14L; I14F; I14V; I14M; (4) V18L; V18F; V18I; (5) E12A; (6) R65D; (7) R86K; R86T; R86M; (8) D87E; D87K; D87R; D87G; D87S; (9) T88S, T88I; T88K; T88R; (10) K89R; K89T; K89M; (11) L101F; L101I; L101Y; L101H; L101N; (12) K104R; K104T; K104M; (13) K105T; K105A; K105R; K105E; (14) F107L; F107I; F107V; F107M; and (15) R108K; R108T; R108M, which substitutions cause an altered affinity for one or both of IL-13Rα1 and IL-13Rα2. In other embodiments, modified residues are at two or more, three or more, four or more, five or more, and not more than 14 amino acids within the combined set of contact residues defined above.

In some embodiments an IL-13 superkine sequence comprises a set of amino acid substitutions relative to SEQ ID NO:1 selected from [L10D, R11I, V18I, R86K, D87K, K89R, R108K]; [L10A, R86T, D87G, T88K, K89R, L101N, K104R, K105A, R108K]; [L10V, K89R, L101N, K105E, R108T]; [R11S, I14M, T88S, L101N, K105A, R108K]; [L10H, R11L, V18I, R86K, D87E, K89R, L101N, K105T, R108K]; [L10H, R11L, V18I, R86M, K89R, R108K]; [L10H, R86T, D87G, T88R, R108K]; [L10H, R86M, T88S, K89R, L101N, K104R, K105A, R108K]; [L10A, V18F, R86K, K89R, L101I, K104R, R108K]. In some such embodiments the IL-13 superkine sequence comprises the set of amino acid substitutions [L10H, R86T, D87G, T88R, R108K], which may be referred to in the Examples as C11. In some such embodiments the IL-13 superkine sequence comprises the set of amino acid substitutions L10A, V18F, R86K, D87K, K89R, L101I, K104R, and R108K, which may be referred to in the figures as D7.

In a CAR sequence the IL-13 superkine may be fused or otherwise joined to a linker sequence that tethers the superkine to the cell. The linker may provide a hinge sequence. The linker may comprise a transmembrane domain connecting the IL-13 superkine to one or more intracellular signaling region of the CAR. Various transmembrane sequences are useful for this purpose, including without limitation those derived from immunoglobulin sequences such as IgG1, IgG4, IgG2, IgG3, etc.; from T cell receptor sequences, from CD3, CD4, CD8, CD28 sequences, etc. The intracellular signaling region comprises one or more signaling domain(s). The signaling region generally includes at least a functional signaling domain from the zeta chain of the human CD3 complex (CD3ζ). Additional signaling domains are optionally included and may comprise, without limitation, one or more of a CD28 signaling domain, a CD137 signaling domain, an OX-40 signaling domain, an ICOS signaling domain, a DAP10 signaling domain, etc. or a combination thereof. The signaling domains may be human.

In some embodiments, a CAR polypeptide comprising an IL-13 superkine is provided. In some embodiments a nucleic acid encoding a CAR polypeptide comprising an IL-13 superkine is provided.

In some embodiments a TAC polypeptide comprising an IL-13 superkine is provided. In some embodiments a nucleic acid encoding a TAC polypeptide comprising an IL-13 superkine is provided.

In some embodiments an ACTR polypeptide system comprising an IL-13 superkine is provided. The IL-13 superkine may be fused to an antibody Fc region with high affinity for CD16 to enhance the interaction with the CAR component of the system. In some embodiments a nucleic acid encoding an ACTR polypeptide system comprising an IL-13 superkine is provided.

In some embodiments a BiTE polypeptide comprising an IL-13 superkine is provided. The IL-13 superkine may be joined to a CD3-binding scFv domain that bridges T cells to a tumour. In some embodiments a nucleic acid encoding a BiTE polypeptide comprising an IL-13 superkine is provided.

Nucleic acid coding sequences may be operably joined to control regions for expression in a T cells. The nucleic acid may be provided in a vector for transfer to a T cell of interest. Vectors of interest include, without limitation, lentivirus vectors, sleeping beauty vectors, plasmid vectors, retrovirus vectors, and the like.

In some embodiments of the invention a population of genetically modified immune cells is provided, which cells are engineered to express a targeting construct comprising an IL-13 superkine. Such cells may be referred to as superkine targeting immune-cells, including targetin T cells, targeting NK cells, etc. In some embodiments the T cells are CD8+ T cells. In some embodiments the T cells are CD4+ T cells. In other embodiments the targeting immune cells are NK cells, e.g. modified NK cell lines, peripheral blood NK cells, iPSC derived NK cells, and the like. The immune cells may be human, and may be autologous or allogeneic relative to an individual selected for treatment. The immune cells may be further modified to enhance therapeutic potential, e.g. by the introduction of one or more transgenes encoding cytokines, co-stimulatory ligands, etc. The immune cells may be modified by deletion of glucocorticoid receptor sites to provide resistance to glucocorticoid treatment. The immune cells may be isolated, and manipulated, expanded, etc. in culture. The population of immune cells may be provided as a pharmaceutical formulation, optionally in a unit dose formulation.

Embodiments of the invention include methods of cancer immunotherapy, which methods comprise administering to a patient in need thereof nucleic acids, vectors, or genetically modified T cells that encode or comprise a T cell targeting construct comprising an IL-13 superkine. Cancers of interest for treatment include hematologic cancers, e.g. leukemias and lymphomas, and solid tumors, e.g. glioblastoma, medulloblastoma, breast cancer, head and neck cancer, kidney cancer, ovarian cancer, Kaposi's sarcoma, acute myelogenous leukemia, B-lineage malignancies, colorectal, pancreatic, kidney, mesothelioma, etc.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Crystal structure of the IL-13 ternary ectodomain complex. Site II and site III interfaces are indicated with a red circle. Left panels show a zoom in on the interfaces where representative positions mutated in the site II (helix A and D) and site III (C-D loop) are highlighted in orange. IL-13 is in orange, IL-13Rα1 is in purple and IL-4Rα1 is in cyan.

FIG. 2. Comparative analysis of the IL-13Rα1- and the IL-13Rα2-selective IL-13 variants (a) Human IL-13 and IL-13Rα1 and IL-13Rα2 selective variants sequences are given for the indicated residue numbers. Kinetic and affinity parameters were determined by surface plasmon resonance. (b) Histogram representation of the normalized K_{D} binding affinity values for IL-13Rα1 (purple) and IL-13Rα2 (orange) selective variants. IL-13 wt K_{D} value was normalized to one and the rest of the values were changed accordingly.

FIG. 3. Signaling activation induced by the IL-13Rα1- and the IL-13Rα2-selective IL-13 variants. (a) The IL-13 responsive cell line A549 was stimulated with doses ranging from 500 nM to 5E-06 nM of the different IL-13 variants for fifteen minutes. Cells were then fixed and permeabilized with 100% cold methanol and stained with antibody against phosphorylated Stat6. The percentage of the MFI value was used to plot the data. (b) A549 cells were stimulated with 500 nM of the IL-13 variants for the indicated times, fixed, permeabilized and stained with phospho-specific Stat antibodies as indicated above and the ratio P-Stat6/P-Stat3 was plotted against time.

FIG. 4. Functional outcomes induced by the IL-13Rα1- and the IL-13Rα2-selective IL-13 variants. (a) Human monocytes were purified from peripheral blood mononuclear cells and cultured with 50 ng/ml GM-CSF alone or with the indicated doses of the different IL-13 variants Cells were analyzed on day 6 with mAbs against HLA-DR, CD86, CD209. Data (mean and SEM) are from 3 donors. (b) TF-1 cells were seeded in a p96 well plate (100.000 cells/well) and stimulated for five days with the indicated doses of the IL-13 variants. Cells were then washed twice with cold PBS and fixed with 4% PFA. The number of cells in each well was determined by flow cytometry. The experiment was repeated three times and the means and SEMs were plotted versus the concentration of cytokine used.

FIG. 5. Crystal structure of the IL-13 ternary ectodomain complex. (a) Site I is highlighted with a red circle. Left panel show a closer look of the site I interface where amino acids mutated in the C helix are colored in orange. IL-13 is in orange, IL-13Rα1 is in purple and IL-4Rα1 is in cyan. (b) Sequences for human IL-13 and IL-13dn are given for the indicated residue numbers. Kinetic and affinity parameters were determined by surface plasmon resonance

FIG. 6. Analysis of the IL-13dn efficacy in vivo. (a) Schematic flowchart where the doses and times used to test the efficacy of IL-13dn in vivo are indicated. (b) qPCR analysis of the expression levels of the Th2 inflammation markers (Muc5ac, Periostin, Arg1, CHIA, YM1, Fizz1) induced by mouse IL-13 in the presence of the indicated dose of IL-13dn.

FIG. 7. Diagram of 1^{st}, 2^{nd}, 3^{rd} and 4^{th} generation chimeric antigen receptors with an IL-13 superkine.

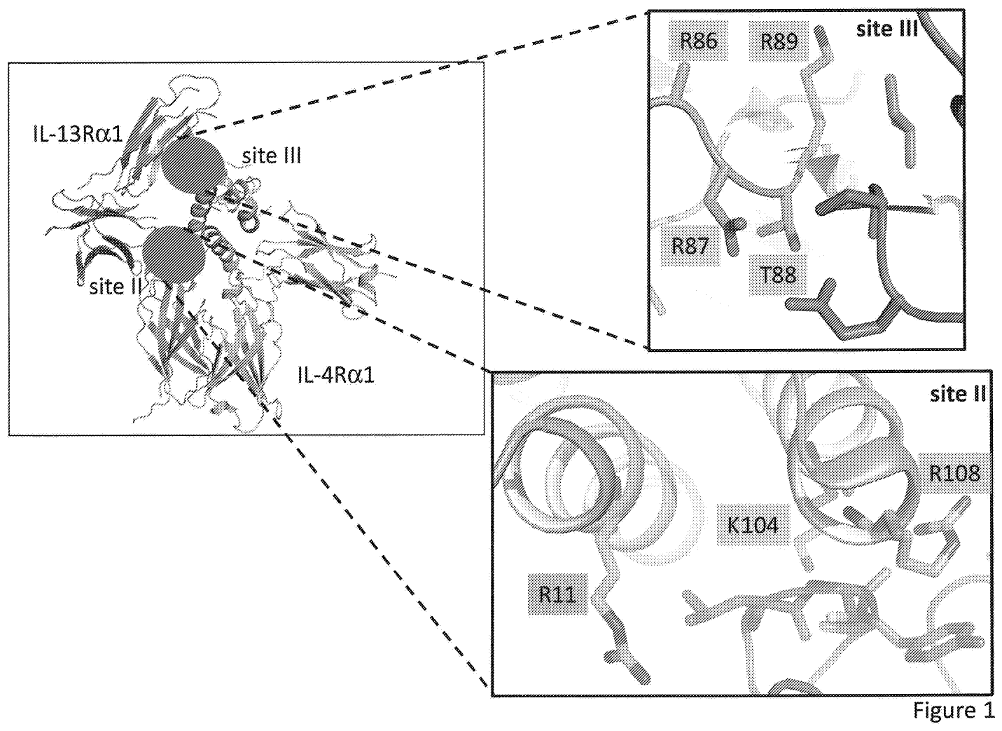

### DEFINITIONS

In the description that follows, a number of terms conventionally used in the field of cell culture are utilized. In order to provide a clear and consistent understanding of the specification and claims, and the scope to be given to such terms, the following defintions are provided.

An “IL-13 superkine”, as used herein, refers to IL-13 polypeptides comprising amino acid substitutions that alter the affinity of the polypeptide to it's receptors, which are one or more of L-13Rα2, IL-13Rα1 and IL-4R. Amino acid modifications may be made at one or more of the amino acids within the set of contact residues that interact with IL-13Rα1 and IL-13Rα2, which residues include, without limitation, L10, R11, I14, V18, R86, D87, T88, K89, L101, K104, K105 and R108 (for reference purposes the sequence of wild-type human IL-13 is provided herein as SEQ ID NO:1, to which the numbering of amino acids will refer). In other embodiments, modified residues are at two or more, three or more, four or more, five or more, and not more than 14 amino acids within the combined set of contact residues defined above. The IL-13 interface that contacts IL-13Rα1 and IL-13Rα2 is the same, and thus there can be overlap in the altered residues that control affinity for these two receptors. In some embodiments one or more of the native amino acid residues L10, R11, I14, V18, R86, D87, T88, K89, L101, K104, K105, F107, and R108 is substituted, and provides for an altered affinity for one or both of IL-13Rα1 and IL-13Rα2.

According to the present invention, amino acid modifications include any naturally occurring or man-made amino acid modifications known or later discovered in the field. In some embodiments, amino acid modifications include any naturally occurring mutation, e.g., substitution, deletion, addition, insertion, etc. In some other embodiments, amino acid modifications include replacing existing amino acid with another amino acid, e.g., a conservative equivalent thereof. In yet some other embodiments, amino acid modifications include replacing one or more existing amino acids with non-natural amino acids or inserting one or more non-natural amino acids. In still some other embodiments, amino acid modifications include at least 1, 2, 3, 4, 5, 6, 8, 10, 12 or 14 amino acid mutations or changes. In some exemplary embodiments, one or more amino acid modifications can be used to alter properties of the IL-13 polypeptide, e.g., affecting the binding activity and/or specificity, etc. Techniques for in vitro mutagenesis of cloned genes are known in the art and described in the Examples herein.

The terms “inhibitors,” “antagonists” refer to an agent that reduces the effective biological activity of IL-13 present in the system, e.g. an animal, a tissue, an in vitro culture system, etc., for example endogenous IL-13 in an individual, usually by interfering with the interaction between IL-13 and one or more of its receptors. For example, an antagonist of the invention may bind tightly to the IL-13Rα1 receptor, but have low affinity to IL-13Rα2, so that it is not “trapped” by that receptor. Antagonists may also have ablated binding to IL-4Rα, to prevent signaling through that receptor. For development purposes the binding may be performed under experimental conditions, e.g. using isolated proteins as binding partners, using portions of proteins as binding partners, using yeast display of proteins or portions of proteins as binding partners, and the like.

Altered affinity for IL-13Rα2, relative to the native human IL-13 protein. The human interleukin 13 receptor, alpha 2 (IL13RA2) may be referenced with the genetic sequence of Genbank accession number NM_000640. The predicted 380-amino acid protein contains a putative signal sequence, an extracellular region with a fibronectin-like domain and typical cytokine receptor motifs, a transmembrane domain, and a short intracellular tail. Amino acid substitutions that provide for altered Rα2 affinity include without limitation (1) L10H; L10A; (2) R11L; (4) V18I; (7) R86M; R86K; R86T; (8) D87K; D87G; (9) T88S; T88R, T88K; (10) K89R; (11) L101N; (12) K104R; (13) K105A; K105E; (14) R108K.

IL13 binds with high affinity to IL13RA1, which induces heterodimerization with IL4R to form a complex, or alternatively, IL13 may bind with even greater affinity to IL13RA2, which fails to induce a signal, indicating that it acts as a decoy receptor. The C-terminal tails of the IL4 and IL13 receptor subunits interact with tyrosine kinases of the Janus kinase family (e.g., JAK1), leading to interaction with STAT6, which binds to consensus sequences in the promoters of IL4- and IL13-regulated genes.

Affinity for IL-13Rα2 to wild-type IL-13 is high, and therefore only modest increases in affinity will be found in polypeptides of the invention, for example equivalent, 2-fold increase, 3-fold increase, 5-fold, 10-fold increase of kinetic K_{D}. In some embodiments, the increase in affinity for IL-13Rα2 as compared to wild-type IL-13 is 2-fold increase increase of kinetic K_{D}. In some embodiments, the increase in affinity for IL-13Rα2 as compared to wild-type IL-13 is 3-fold increase increase of kinetic K_{D}. In some embodiments, the increase in affinity for IL-13Rα2 as compared to wild-type IL-13 is, 5-fold increase of kinetic K_{D}. In some embodiments, the increase in affinity for IL-13Rα2 as compared to wild-type IL-13 is 10-fold increase of kinetic K_{D}. For example, polypeptide C11 (SEQ ID NO:18 or SEQ ID NO:35) and D7 (SEQ ID NO:20 or SEQ ID NO:37) show increased binding to IL-13Rα2 and decreased binding to L-13Rα1, and have the set of amino acid substitutions [L10H, R86T, D87G, T88R, R108K] and [L10A, V18F, R86K, D87K, K89R, L101I, K104R, R108K], respectively. In some embodiments, polypeptide with an increase in affinity for IL-13Rα2 as compared to wild-type IL-13 comprises substitutions L10A, V18F, R86K, D87K, K89R, L101I, K104R, and R108K. In some embodiments, polypeptide with an increase in affinity for IL-13Rα2 as compared to wild-type IL-13 comprises substitutions L10H, R86T, D87G, T88R, R108K. In some embodiments, polypeptide with an increase in affinity for IL-13Rα2 as compared to wild-type IL-13 comprises C11 (SEQ ID NO: 18 or SEQ ID NO:35). In some embodiments, polypeptide with an increase in affinity for IL-13Rα2 as compared to wild-type IL-13 comprises D7 (SEQ ID NO:20 or SEQ ID NO:37).

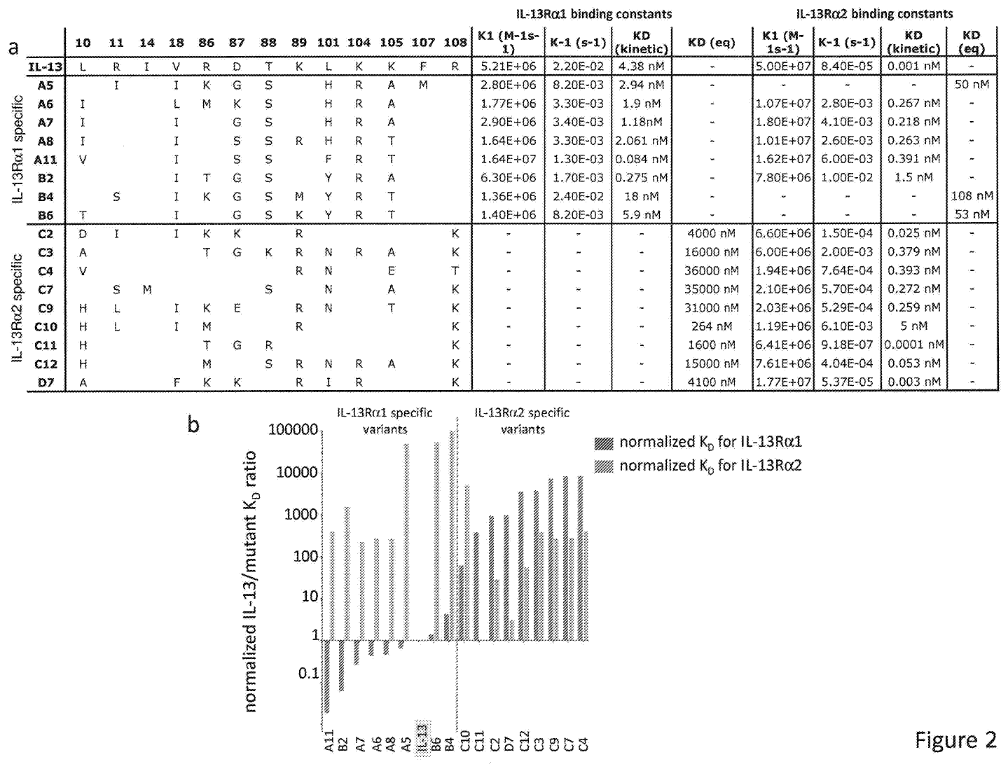

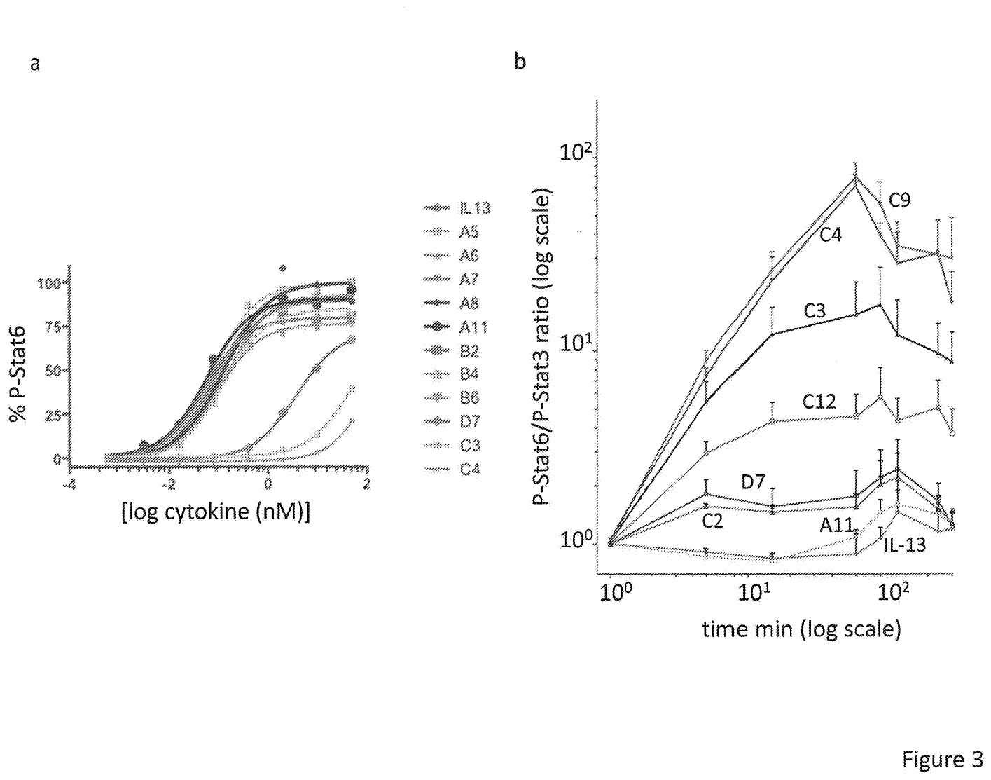

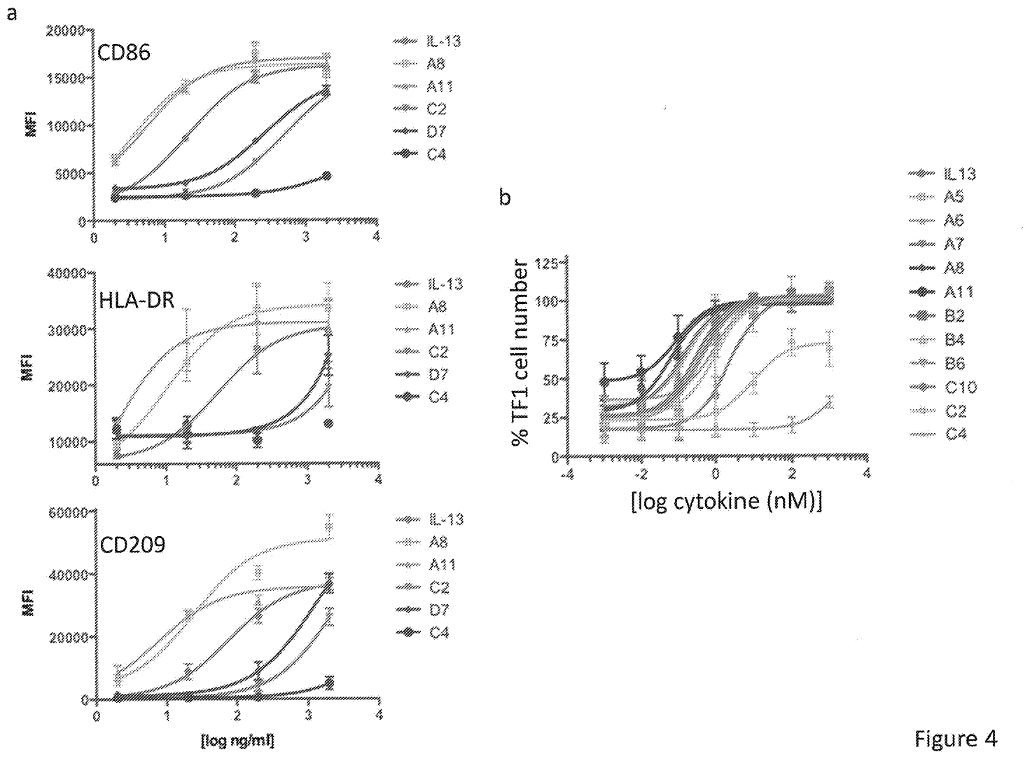

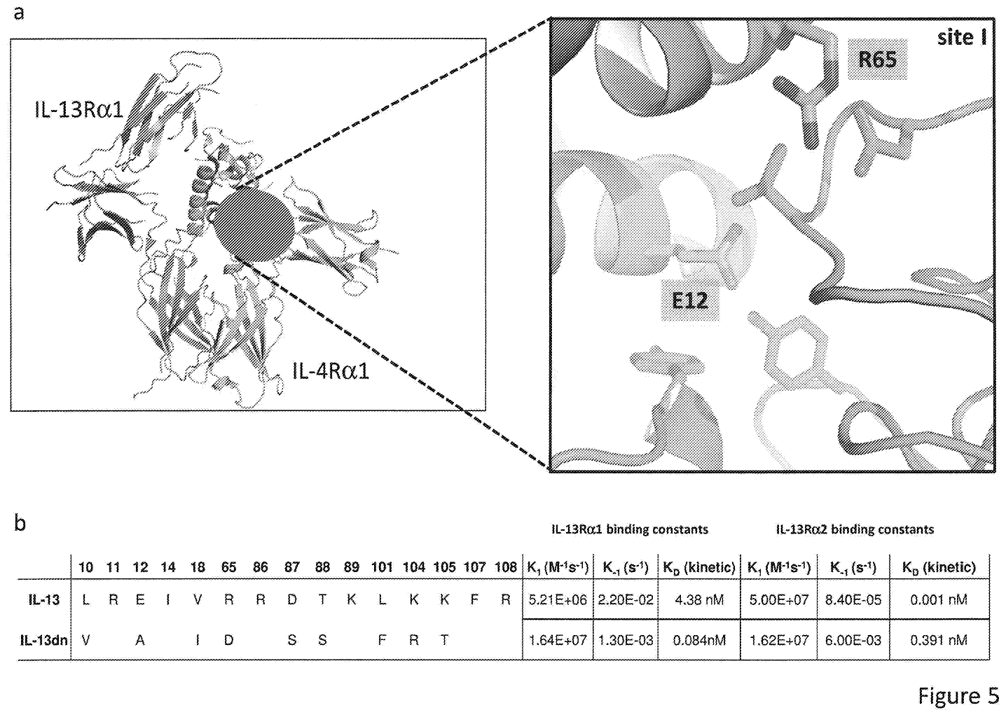

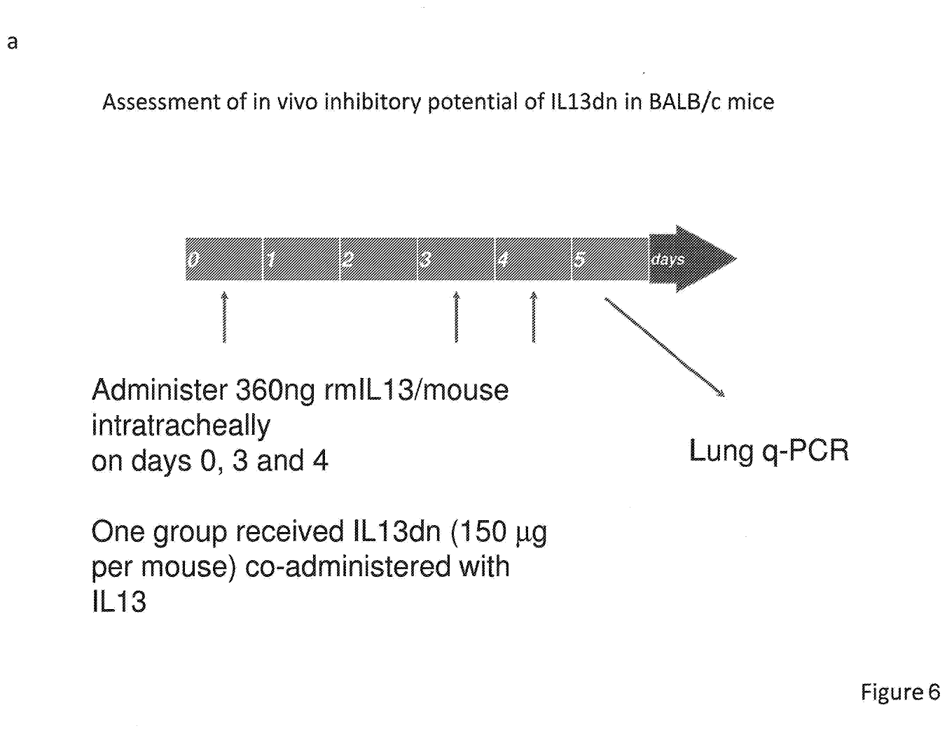

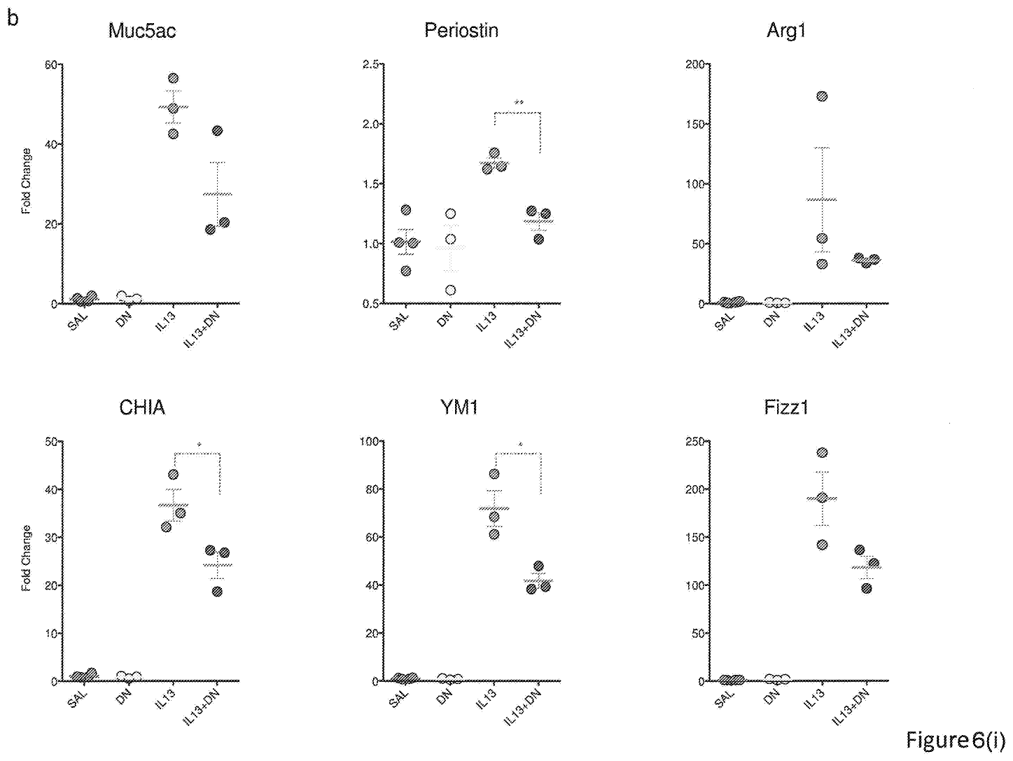

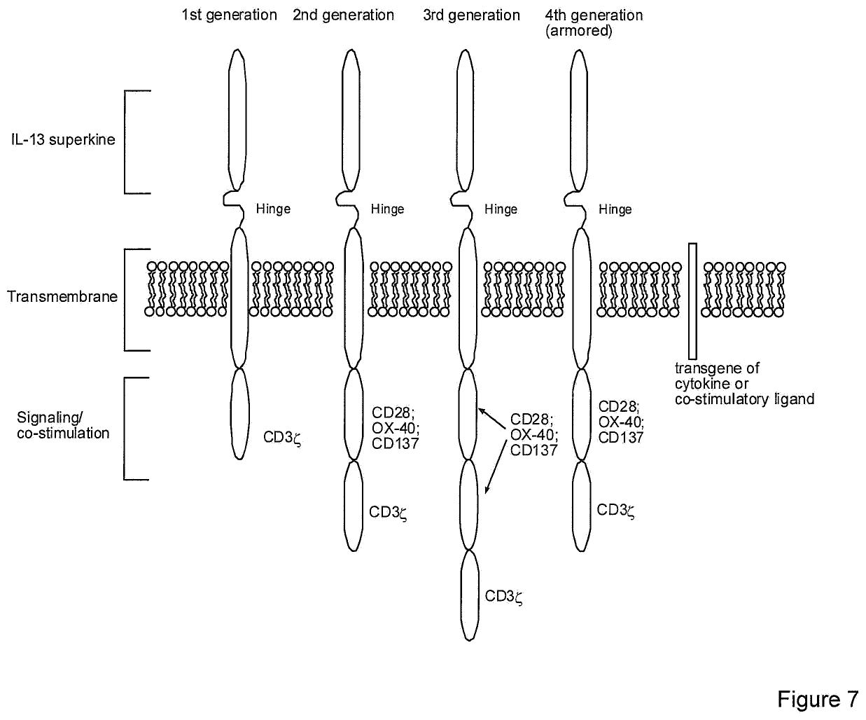

Altered affinity for IL-13Rα1 relative to the native human IL-13 protein. The human interleukin 13 receptor, alpha 1 (IL13RA1) may be referenced with the genetic sequence of Genbank accession number NM_001560. It is a protein of 424 amino acid residues, containing a putative signal sequence and transmembrane domain, which is a low-affinity receptor. Amino acid substitutions that provide for altered Rα1 affinity include without limitation (1) L10I, L10V; (4) V18I; (7) R86K, R86M; (8) D87G, D87S; (9) T88S; (10) K89R, K89M; (11) L101H, L101Y; (12) K104R; and (13) K105A; K105T. In some embodiments, amino acid substitutions that provide for altered IL-13Rα1 affinity include without limitation those provided in FIG. 2.

Decreases in affinity may be modest, for example equivalent, 2-fold decrease, 3-fold decrease, 5-fold decrease of kinetic K_{D}. Decrease in affinity can also be greater than about 10-fold, greater than about 10²-fold, greater than about 10³-fold or more. In some embodiments, the decrease in affinity for IL-13Rα1 is about a 2-fold decrease of kinetic K_{D}. In some embodiments, the decrease in affinity for IL-13Rα1 is about a 3-fold decrease of kinetic K_{D}. In some embodiments, the decrease in affinity for IL-13Rα1 is about a 4-fold decrease of kinetic K_{D}. In some embodiments, the decrease in affinity for IL-13Rα1 is a 5-fold decrease of kinetic K_{D}. In some embodiments, the decrease in affinity for IL-13Rα1 is a greater than about 10-fold decrease of kinetic K_{D}. In some embodiments, the decrease in affinity for IL-13Rα1 is a greater than about 10²-fold decrease of kinetic K_{D}. In some embodiments, the decrease in affinity for IL-13Rα1 is a greater than about 10³-fold decrease of kinetic K_{D}. In some embodiments, the decrease in affinity for IL-13Rα1 is a greater than about 10⁴-fold decrease of kinetic K_{D}. For example, polypeptide B4 (SEQ ID NO:9) provides a decreased affinity to IL-13Rα1, and has the set of amino acid substitutions [R11S, V18I, R86K, D87G, T88S, K89M, L101Y, K104R, K105T]. Polypeptide C4 provides a decreased affinity to IL-13Rα1, and has the set of amino acid substitutions [L10V, K89R, L101N, K105E, R108T]. In some embodiments, variants that provide for decreased Rα1 affinity include without limitation those provided in FIG. 2.

The binding properties of a binding agent may be measured by any method, e.g., one of the following methods: BIACORE™ analysis, Enzyme Linked Immunosorbent Assay (ELISA), x-ray crystallography, sequence analysis and scanning mutagenesis. The ability of a protein to neutralize and/or inhibit one or more IL-13-associated activities may be measured by the following methods: assays for measuring the proliferation of an IL-13 dependent cell line, e.g. TFI; assays for measuring the expression of IL-13-mediated polypeptides, e.g., flow cytometric analysis of the expression of CD23; assays evaluating the activity of downstream signaling molecules, e.g., STAT6; assays evaluating production of tenascin; assays testing the efficiency of an described herein to prevent asthma in a relevant animal model, e.g., the cynomolgus monkey, and other assays. An IL-13 polypeptide can have a statistically significant effect in one or more of these assays. Exemplary assays for binding properties include the following.

The binding interaction of an IL-13 polypeptide and a target (e.g., receptor) can be analyzed using surface plasmon resonance (SPR). SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (Kd), and kinetic parameters, including Kon and Koff, for the binding of a molecule to a target. Such data can be used to compare different molecules. Information from SPR can also be used to develop structure-activity relationships (SAR). For example, the kinetic and equilibrium binding parameters of different molecule can be evaluated. Variant amino acids at given positions can be identified that correlate with particular binding parameters, e.g., high affinity and slow Koff. This information can be combined with structural modeling (e.g., using homology modeling, energy minimization, or structure determination by x-ray crystallography or NMR). As a result, an understanding of the physical interaction between the protein and its target can be formulated and used to guide other design processes.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the abbreviations for the genetically encoded L-enantiomeric amino acids used in the disclosure methods are conventional and are as follows in Table 1. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, i.e., an alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

TABLE 1
Amino acid abbreviations

| Amino Acid | One-Letter Symbol | Common Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

“Hydrophilic Amino Acid” refers to an amino acid exhibiting a hydrophobicity of less than zero according to the normalized consensus hydrophobicity scale of Eisenberg et al., 1984, J. Mol. Biol. 179: 125-142. Genetically encoded hydrophilic amino acids include Thr (T), Ser (S), His (H), Glu (E), Asn (N), Gln (Q), Asp (D), Lys (K) and Arg (R).

“Acidic Amino Acid” refers to a hydrophilic amino acid having a side chain pK value of less than 7. Acidic amino acids typically have negatively charged side chains at physiological pH due to loss of a hydrogen ion. Genetically encoded acidic amino acids include Glu (E) and Asp (D).

“Basic Amino Acid” refers to a hydrophilic amino acid having a side chain pK value of greater than 7. Basic amino acids typically have positively charged side chains at physiological pH due to association with hydrogen ion. Genetically encoded basic amino acids include His (H), Arg (R) and Lys (K).

“Polar Amino Acid” refers to a hydrophilic amino acid having a side chain uncharged at physiological pH, but which has at least one bond in which the pair of electrons shared in common by two atoms is held more closely by one of the atoms. Genetically encoded polar amino acids include Asn (N), Gln (Q), Ser (S) and Thr (T).

“Hydrophobic Amino Acid” refers to an amino acid exhibiting a hydrophobicity of greater than zero according to the normalized consensus hydrophobicity scale of Eisenberg, 1984, J. Mol. Biol. 179:125-142. Exemplary hydrophobic amino acids include Ile (I), Phe (F), Val (V), Leu (L), Trp (W), Met (M), Ala (A), Gly (G), Tyr (Y), Pro (P), and proline analogues.

“Aromatic Amino Acid” refers to a hydrophobic amino acid with a side chain having at least one aromatic or heteroaromatic ring. The aromatic or heteroaromatic ring may contain one or more substituents such as —OH, —SH, —CN, —F, —CI, —Br, —I, —NO2, —NO, —NH2, —NHR, —NRR, —C(O)R, —C(O)OH, —C(O)OR, —C(O)NH2, —C(O)NHR, —C(O)NRR and the like where each R is independently (CI-C6) alkyl, substituted (CI-C6) alkyl, (CI-C6) alkenyl, substituted (CI-C6) alkenyl, (CI-C6) alkynyl, substituted (CI-C6) alkynyl, (CI-C21)) aryl, substituted (C5-020) aryl, (C6-C26) alkaryl, substituted (C6-C26) alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe (F), Tyr (Y) and Trp (W).

“Nonpolar Amino Acid” refers to a hydrophobic amino acid having a side chain uncharged at physiological pH and which has bonds in which the pair of electrons shared in common by two atoms is generally held equally by each of the two atoms (i.e., the side chain is not polar). Genetically encoded apolar amino acids include Leu (L), Val (V), Ile (I), Met (M), Gly (G) and Ala (A).

“Aliphatic Amino Acid” refers to a hydrophobic amino acid having an aliphatic hydrocarbon side chain. Genetically encoded aliphatic amino acids include Ala (A), Val (V), Leu (L) and Ile (I).

The term “non-naturally” with regard to amino acids can include any amino acid molecule not included as one of the 20 amino acids listed in Table 1 above as well as any modified or derivatized amino acid known to one of skill in the art. Non-naturally amino acids can include but are not limited to β-alanine, α-amino butyric acid, γ-amino butyric acid, γ-(aminophenyl) butyric acid, α-amino isobutyric acid, ε-amino caproic acid, 7-amino heptanoic acid, β-aspartic acid, aminobenzoic acid, aminophenyl acetic acid, aminophenyl butyric acid, γ-glutamic acid, cysteine (ACM), ε-lysine, methionine sulfone, norleucine, norvaline, ornithine, d-ornithine, p-nitro-phenylalanine, hydroxy proline, 1,2,3,4,-tetrahydroisoquinoline-3-carboxylic acid, and thioproline.

The term “variant” or “variants”, with regard to polypeptides, such as capsid polypeptides refers to a polypeptide sequence differing by at least one amino acid from a parent polypeptide sequence, for example wildtype IL-13 (SEQ ID NO:1). Amino acids also include naturally occurring and non-naturally occurring amino acids as well as derivatives thereof. Amino acids also include both D and L forms.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having disease. Subjects may be human, but also include other mammals, particularly those mammals useful as laboratory models for human disease, e.g. mouse, rat, etc.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as disease cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's disease cell, e.g., a sample comprising polynucleotides and/or polypeptides that is obtained from a patient's disease cell (e.g., a cell lysate or other cell extract comprising polynucleotides and/or polypeptides); and a sample comprising disease cells from a patient. A biological sample comprising a disease cell from a patient can also include non-diseased cells.

The term “diagnosis” is used herein to refer to the identification of a molecular or pathological state, disease or condition.

The term “prognosis” is used herein to refer to the prediction of the likelihood of death or progression, including recurrence, spread, and drug resistance. The term “prediction” is used herein to refer to the act of foretelling or estimating, based on observation, experience, or scientific reasoning. In one example, a physician may predict the likelihood that a patient will survive.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of an atopic disorder or tumor in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

“Concomitant administration” of a known disease therapeutic drug with a pharmaceutical composition of the present invention means administration of the drug and IL-13 polypeptide at such time that both the known drug and the composition of the present invention will have a therapeutic effect. Such concomitant administration may involve concurrent (i.e. at the same time), prior, or subsequent administration of the drug with respect to the administration of a compound of the invention. A person of ordinary skill in the art would have no difficulty determining the appropriate timing, sequence and dosages of administration for particular drugs and compositions of the present invention.

Cancers of interest include carcinomas, e.g. colon, prostate, breast, melanoma, ductal, endometrial, stomach, dysplastic oral mucosa, invasive oral cancer, non-small cell lung carcinoma, transitional and squamous cell urinary carcinoma, etc.; neurological malignancies, e.g. neuroblastoma, gliomas, gliobastoma multiforme, etc.; hematological malignancies, e.g. childhood acute leukemia, non-Hodgkin's lymphomas, chronic lymphocytic leukemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; sarcomas, melanomas, adenomas; benign lesions such as papillomas, and the like.

The present compositions and methods are applicable to brain tumors, particularly glioblastoma. In general, the goals of brain tumor treatments are to remove as many tumor cells as possible, e.g. with surgery, kill as many of the cells left behind after surgery as possible with radiation and/or chemotherapy, and put remaining tumor cells into a nondividing, quiescent state for as long as possible with radiation and chemotherapy. Careful imaging surveillance is a crucial part of medical care, because tumor regrowth requires alteration of current treatment, or, for patients in the observation phase, restarting treatment.

Brain tumors are classified according to the kind of cell from which the tumor seems to originate. Diffuse, fibrillary astrocytomas are the most common type of primary brain tumor in adults. These tumors are divided histopathologically into three grades of malignancy: World Health Organization (WHO) grade II astrocytoma, WHO grade III anaplastic astrocytoma and WHO grade IV glioblastoma multiforme (GBM). WHO grade II astrocytomas are the most indolent of the diffuse astrocytoma spectrum. Astrocytomas display a remarkable tendency to infiltrate the surrounding brain, confounding therapeutic attempts at local control. These invasive abilities are often apparent in low-grade as well as high-grade tumors.

Glioblastoma multiforme is the most malignant stage of astrocytoma, with survival times of less than 2 years for most patients. Histologically, these tumors are characterized by dense cellularity, high proliferation indices, endothelial proliferation and focal necrosis. The highly proliferative nature of these lesions likely results from multiple mitogenic effects. One of the hallmarks of GBM is endothelial proliferation. A host of angiogenic growth factors and their receptors are found in GBMs.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

### DETAILED DESCRIPTION OF THE EMBODIMENTS

Immune cell targeting constructs comprising IL-13 superkine sequences are provided. The superkines have altered affinity for one or more receptors selected from IL-13Rα1, IL-13Rα2 and IL-4R, as described above in detail. Superkines are useful for targeting immune cells to cells, e.g. tumor cells, expressing the at least one receptor, e.g. IL-13α2.

The IL-13 superkine component of the construct may be at least about 50 amino acids in length, at least about 75, at least about 100, at least about 110, at least about 115 amino acids in length, up to the full-length of the wild-type protein at the transmembrane domain, i.e. about 116 amino acids in length. For example, the superkine may be fused to the hinge, transmembrane or signaling domains of a CAR. Exemplary polypeptide sequences are provided in SEQ ID NO:2-SEQ ID NO:38. In some embodiments, the polypeptide sequence is as provided in any one of SEQ ID NO:2 through SEQ ID NO:38. In some embodiments, the polypeptide sequence is SEQ ID NO:2. In some embodiments, the polypeptide sequence is SEQ ID NO:2. In some embodiments, the polypeptide sequence is SEQ ID NO:3. In some embodiments, the polypeptide sequence is SEQ ID NO:4. In some embodiments, the polypeptide sequence is SEQ ID NO:5. In some embodiments, the polypeptide sequence is SEQ ID NO:6. In some embodiments, the polypeptide sequence is SEQ ID NO:7. In some embodiments, the polypeptide sequence is SEQ ID NO:8. In some embodiments, the polypeptide sequence is SEQ ID NO:9. In some embodiments, the polypeptide sequence is SEQ ID NO:10. In some embodiments, the polypeptide sequence is SEQ ID NO:11. In some embodiments, the polypeptide sequence is SEQ ID NO:12. In some embodiments, the polypeptide sequence is SEQ ID NO:13. In some embodiments, the polypeptide sequence is SEQ ID NO:14. In some embodiments, the polypeptide sequence is SEQ ID NO:15. In some embodiments, the polypeptide sequence is SEQ ID NO:16. In some embodiments, the polypeptide sequence is SEQ ID NO:17. In some embodiments, the polypeptide sequence is SEQ ID NO:18. In some embodiments, the polypeptide sequence is SEQ ID NO:19. In some embodiments, the polypeptide sequence is SEQ ID NO:20. In some embodiments, the polypeptide sequence is SEQ ID NO:21. In some embodiments, the polypeptide sequence is SEQ ID NO:22. In some embodiments, the polypeptide sequence is SEQ ID NO:23. In some embodiments, the polypeptide sequence is SEQ ID NO:24. In some embodiments, the polypeptide sequence is SEQ ID NO:25. In some embodiments, the polypeptide sequence is SEQ ID NO:26. In some embodiments, the polypeptide sequence is SEQ ID NO:27. In some embodiments, the polypeptide sequence is SEQ ID NO:28. In some embodiments, the polypeptide sequence is SEQ ID NO:29. In some embodiments, the polypeptide sequence is SEQ ID NO:30. In some embodiments, the polypeptide sequence is SEQ ID NO:31. In some embodiments, the polypeptide sequence is SEQ ID NO:32. In some embodiments, the polypeptide sequence is SEQ ID NO:33. In some embodiments, the polypeptide sequence is SEQ ID NO:34. In some embodiments, the polypeptide sequence is SEQ ID NO:35. In some embodiments, the polypeptide sequence is SEQ ID NO:36. In some embodiments, the polypeptide sequence is SEQ ID NO:37. In some embodiments, the polypeptide sequence is SEQ ID NO:38. In some embodiments, the polypeptide sequence is 90% identical to any one of SEQ ID NO:2 through SEQ ID NO:38. In some embodiments, the polypeptide sequence is 95% identical to any one of SEQ ID NO:2 through SEQ ID NO:38. In some embodiments, the polypeptide seqeunce is 98% identical to any one of SEQ ID NO:2 through SEQ ID NO:38. In some embodiments, the polypeptide sequence is 99% identical to any one of SEQ ID NO:2 through SEQ ID NO:38. In some embodiments, any one of SEQ ID NO:2 through SEQ ID NO:38 are linked to an an IL-13 superkine immune cell targeting construct. In some embodiments, SEQ ID NO:2 is linked to an an IL-13 superkine immune cell targeting construct. In some embodiments, SEQ ID NO:3 is linked to an an IL-13 superkine immune cell targeting construct. In some embodiments, SEQ ID NO:4 is linked to an an IL-13 superkine immune cell targeting construct. In some embodiments, SEQ ID NO:5 is linked to an an IL-13 superkine immune cell targeting construct. In some embodiments, SEQ ID NO:6 is linked to an an IL-13 superkine immune cell targeting construct. In some embodiments, SEQ ID NO:7 is linked to an an IL-13 superkine immune cell targeting construct. In some embodiments, SEQ ID NO:8 is linked to an an IL-13 superkine immune cell targeting construct. In some embodiments, SEQ ID NO:9 is linked to an an IL-13 superkine immune cell targeting construct. In some embodiments, SEQ ID NO:10 is linked to an an IL-13 superkine immune cell targeting construct. In some embodiments, SEQ ID NO:11 is linked to an an IL-13 superkine immune cell targeting construct. In some embodiments, SEQ ID NO:12 is linked to an an IL-13 superkine immune cell targeting construct. In some embodiments, SEQ ID NO:13 is linked to an an IL-13 superkine immune cell targeting construct. In some embodiments, SEQ ID NO:14 is linked to an an IL-13 superkine immune cell targeting construct. In some embodiments, SEQ ID NO:15 is linked to an an IL-13 superkine immune cell targeting construct. In some embodiments, SEQ ID NO:16 is linked to an an IL-13 superkine immune cell targeting construct. In some embodiments, SEQ ID NO:17 is linked to an an IL-13 superkine immune cell targeting construct. In some embodiments, SEQ ID NO:18 is linked to an an IL-13 superkine immune cell targeting construct. In some embodiments, SEQ ID NO:19 is linked to an an IL-13 superkine immune cell targeting construct. In some embodiments, SEQ ID NO:20 is linked to an an IL-13 superkine immune cell targeting construct. In some embodiments, SEQ ID NO:21 is linked to an an IL-13 superkine immune cell targeting construct. In some embodiments, SEQ ID NO:22 is linked to an an IL-13 superkine immune cell targeting construct. In some embodiments, SEQ ID NO:23 is linked to an an IL-13 superkine immune cell targeting construct. In some embodiments, SEQ ID NO:24 is linked to an an IL-13 superkine immune cell targeting construct. In some embodiments, SEQ ID NO:25 is linked to an an IL-13 superkine immune cell targeting construct. In some embodiments, SEQ ID NO:26 is linked to an an IL-13 superkine immune cell targeting construct. In some embodiments, SEQ ID NO:27 is linked to an an IL-13 superkine immune cell targeting construct. In some embodiments, SEQ ID NO:28 is linked to an an IL-13 superkine immune cell targeting construct. In some embodiments, SEQ ID NO:29 is linked to an an IL-13 superkine immune cell targeting construct. In some embodiments, SEQ ID NO:30 is linked to an an IL-13 superkine immune cell targeting construct. In some embodiments, SEQ ID NO:31 is linked to an an IL-13 superkine immune cell targeting construct. In some embodiments, SEQ ID NO:32 is linked to an an IL-13 superkine immune cell targeting construct. In some embodiments, SEQ ID NO:33 is linked to an an IL-13 superkine immune cell targeting construct. In some embodiments, SEQ ID NO:34 is linked to an an IL-13 superkine immune cell targeting construct. In some embodiments, SEQ ID NO:35 is linked to an an IL-13 superkine immune cell targeting construct. In some embodiments, SEQ ID NO:36 is linked to an an IL-13 superkine immune cell targeting construct. In some embodiments, SEQ ID NO:37 is linked to an an IL-13 superkine immune cell targeting construct. In some embodiments, SEQ ID NO:38 is linked to an an IL-13 superkine immune cell targeting construct. In some embodiments, an IL-13 superkine immune cell targeting construct comprises one or more signaling domains derived from CD3-ζ, CD28, DAP10, OX-40, ICOS and CD137. In some embodiments, an IL-13 superkine immune cell targeting construct comprises one or more signaling domains derived from CD3-ζ. In some embodiments, an IL-13 superkine immune cell targeting construct comprises one or more signaling domains derived from CD28. In some embodiments, an IL-13 superkine immune cell targeting construct comprises one or more signaling domains derived from DAP10. In some embodiments, an IL-13 superkine immune cell targeting construct comprises one or more signaling domains derived from OX-40. In some embodiments, an IL-13 superkine immune cell targeting construct comprises one or more signaling domains derived from CD137.In some embodiments an IL-13 peptide of the invention comprises one or more of the amino acids substitutions: (1) L10F, L10I, L10V, L10A, L10D, L10T, L10H; (2) R11S, R11N, R11H, R11L, R11I; (3) I14L, I14F, 114V, I14M; (4) V18L, V18F, V18I; (5) E12A, (6) R65D, (7) R86K, R86T, R86M; (8) D87E, D87K, D87R, D87G, D87S; (9) T88I, T88K, T88R; (10) K89R, K89T, K89M; (11) L101F, L101I, L101Y, L101H, L101N; (12) K104R, K104T, K104M; (13) K105T, K105A, K105R, K105E; (14) F107L, F107I, F107V, F107M; and (15) R108K, R108T, R108M, which substitutions cause an altered affinity for one or both of IL-13Rα1 and IL-13Rα2. In other embodiments, modified residues are at two or more, three or more, four or more, five or more, and not more than 14 amino acids within the combined set of contact residues defined above. As described in International Patent Publication WO 2013/112871, the disclosure of which is incorporated by reference herein in its entirety. In some embodiments, amino acid substitutions include without limitation those provided in FIG. 2.

Sets of modifications may include the following specific changes: (1) L10H; L10A; (2) R11L; (4) V18I; (7) R86M; R86K; R86T; (8) D87K; D87G; (9) T88R, T88S; T88K; (10) K89R; (11) L101N; (12) K104R; (13) K105A; K105E; (14) R108K. In some embodiments, the modification includes any one of the recited specific changes. In some embodiments, the modification includes L10H. In some embodiments, the modification includes L10A. In some embodiments, the modification includes R11L. In some embodiments, the modification includes V18I. In some embodiments, the modification includes R86M. In some embodiments, the modification includes R86K. In some embodiments, the modification includes R86T. In some embodiments, the modification includes D87K. In some embodiments, the modification includes D87G. In some embodiments, the modification includes T88R. In some embodiments, the modification includes T88S. In some embodiments, the modification includes T88K. In some embodiments, the modification includes K89R. In some embodiments, the modification includes L101N. In some embodiments, the modification includes K104R. In some embodiments, the modification includes K105A. In some embodiments, the modification includes K105E. In some embodiments, the modification includes R108K. In some embodiments, the polypeptide comprising the one or more modifications is linked to an an IL-13 superkine immune cell targeting construct. In some embodiments, an IL-13 superkine immune cell targeting construct comprises one or more signaling domains derived from CD3-ζ, CD28, DAP10, OX-40, ICOS and CD137. In some embodiments, an IL-13 superkine immune cell targeting construct comprises one or more signaling domains derived from CD3-ζ. In some embodiments, an IL-13 superkine immune cell targeting construct comprises one or more signaling domains derived from CD28. In some embodiments, an IL-13 superkine immune cell targeting construct comprises one or more signaling domains derived from DAP10. In some embodiments, an IL-13 superkine immune cell targeting construct comprises one or more signaling domains derived from OX-40. In some embodiments, an IL-13 superkine immune cell targeting construct comprises one or more signaling domains derived from CD137. In some embodiments, amino acid substitutions include without limitation those provided in FIG. 2.

Specific sets of modifications that provide for greater selectivity in binding to IL-13Rα2 v IL-13Rα1 relative to a native IL-13 sequence may include, without limitation:
[L10D, R11I, V18I, R86K, D87K, k89R, R108K] (for example, C2, e.g. SEQ ID NO:11 or SEQ ID NO:29)
[L10A, R86T, D87G, T88K, K89R, L101N, K104R, K105A, R108K] (for example, C3, e.g. SEQ ID NO:12 or SEQ ID NO:30)
[L10V, K89R, L101N, K105E, R108T] (for example, C4, e.g. SEQ ID NO:13 or SEQ ID NO:31)
[R11S, I14M, T88S, L101N, K105A, R108K] (for example, C7, e.g. SEQ ID NO:14 or SEQ ID NO:32)
[L10H, R11L, V18I, R86K, D87E, K89R, L101N, K105T, R108K] (C9, e.g. SEQ ID NO:33)
[L10H, R86T, D87G, T88R, R108K] (C11 e.g. SEQ ID NO:18 or SEQ ID NO:35)
[L10A, V18F, R86K, D87K, K89R, L101I, K104R, R108K] (D7, e.g. SEQ ID NO:20 or SEQ ID NO:37)
[L10T/D; R11I; V18I; R86K; D87K/G; T88S; K89R; L101Y; K104R; K105T; R108K]
[L10A/V; R86T; D87G; T88K; K89R; L101N; K104R; K105A/E; R108K/T]

In some embodiments, the set of modifications comprises L10V, K89R, L101N, K105E, R108T. In some embodiments, the set of modifications comprises R11S, I14M, T88S, [symbol] , K105A, and R108K (C7, e.g. SEQ ID NO:15 or SEQ ID NO:32). In some embodiments, the set of modifications comprises L10H, R11L, V18I, R86K, D87E, K89R, L101N, K105T, and R108K (C9, e.g. SEQ ID NO:16 or SEQ ID NO:33). In some embodiments, the set of modifications comprises L10H, R86T, D87G, T88R, and R108K (C11 e.g. SEQ ID NO:18 or SEQ ID NO:35). In some embodiments, the set of modifications comprises L10A, V18F, R86K, D87K, K89R, L101I, K104R, and R108K (D7, e.g. SEQ ID NO:20 or SEQ ID NO:37). In some embodiments, the set of modifications comprises L10T/D, R11I, V18I, R86K, D87K/G, T88S, K89R, L101Y, K104R, K105T, and R108K. In some embodiments, the set of modifications comprises L10T, R11I, V18I, R86K, D87K, T88S, K89R, L101Y, K104R, K105T, and R108K. In some embodiments, the set of modifications comprises L10T, R11I, V18I, R86K, D87G, T88S, K89R, L101Y, K104R, K105T, and R108K. In some embodiments, the set of modifications comprises L10D, R11I, V18I, R86K, D87K, T88S, K89R, L101Y, K104R, K105T, and R108K. In some embodiments, the set of modifications comprises L10D, R11I, V18I, R86K, D87G, T88S, K89R, L101Y, K104R, K105T, R108K. In some embodiments, the set of modifications comprises L10A/V, R86T, D87G, T88K, K89R, L101N, K104R, K105A/E, and R108K/T. In some embodiments, the set of modifications comprises L10A, R86T, D87G, T88K, K89R, L101N, K104R, K105A, and R108K. In some embodiments, the set of modifications comprises L10A, R86T, D87G, T88K, K89R, L101N, K104R, K105E, and R108K. In some embodiments, the set of modifications comprises L10A, R86T, D87G, T88K, K89R, L101N, K104R, K105A, and R108T. In some embodiments, the set of modifications comprises L10A, R86T, D87G, T88K, K89R, L101N, K104R, K105E, and R108T. In some embodiments, the set of modifications comprises L10V, R86T, D87G, T88K, K89R, L101N, K104R, K105A, and R108K. In some embodiments, the set of modifications comprises L10V, R86T, D87G, T88K, K89R, L101N, K104R, K105E, and R108K. In some embodiments, the set of modifications comprises L10V, R86T, D87G, T88K, K89R, L101N, K104R, K105A, an dR108T. In some embodiments, the set of modifications comprises L10V, R86T, D87G, T88K, K89R, L101N, K104R, K105E, and R108T. In some embodiments, the amino acid seqeunce is 90% identical. In some embodiments, the amino acid sequence is 95% identical. In some embodiments, the amino acid seqeunce is 98% identical. In some embodiments, the amino acid sequence is 99% identical. In some embodiments, the polypeptide comprising the one or more modifications is linked to an an IL-13 superkine immune cell targeting construct. In some embodiments, an IL-13 superkine immune cell targeting construct comprises one or more signaling domains derived from CD3-ζ, CD28, DAP10, OX-40, ICOS and CD137. In some embodiments, an IL-13 superkine immune cell targeting construct comprises one or more signaling domains derived from CD3-ζ. In some embodiments, an IL-13 superkine immune cell targeting construct comprises one or more signaling domains derived from CD28. In some embodiments, an IL-13 superkine immune cell targeting construct comprises one or more signaling domains derived from DAP10. In some embodiments, an IL-13 superkine immune cell targeting construct comprises one or more signaling domains derived from OX-40. In some embodiments, an IL-13 superkine immune cell targeting construct comprises one or more signaling domains derived from CD137. In some embodiments, amino acid substitutions include without limitation those provided in FIG. 2.

Specific sets of modifications that provide for greater selectivity in binding to IL-13Rα1 v IL-13Rα2 relative to a native IL-13 sequence may include, without limitation:
[L10V, V18I, D87S, D88S, L101F, K104R, K105T]
[R11S, V18I, R86K, D87G, T88S, K89M, L101Y, K104R, K105T]
[L10V, V18I, D87S, T88S, L101F, K104R, K105T]
[L10V/I; D87S; T88S; K89R; L101H/F; K104R; K105T]
[L10I; V18I; R86T; D87G; T88S; K89R; L101Y/H; K104R; K105A]
[L10V; V18I; D87S; T88S; L101F; K104R; K105T]
[V18I, R86T, D87G, T88S, L101Y, K104R, K105A]
[R11I, V18I, R86K, D87G, T88S, L101H, K104R, K105A, F107M]
   which substitutions are optionally combined with the substitutions [E12A/G/S, R65D/E].

In some embodiments, the set of modifications comprises L10V, V18I, D87S, D88S, L101F, K104R, and K105T. In some embodiments, the set of modifications comprises R11S, V18I, R86K, D87G, T88S, K89M, L101Y, K104R, and K105T. In some embodiments, the set of modifications comprises L10V, V18I, D87S, T88S, L101F, K104R, and K105T. In some embodiments, the set of modifications comprises L10V/I, D87S, T88S, K89R, L101H/F, K104R, and K105T. In some embodiments, the set of modifications comprises L10I, V18I, R86T, D87G, T88S, K89R, L101Y/H, K104R, and K105A. In some embodiments, the set of modifications comprises L10V, V18I, D87S, T88S, L101F, K104R, and K105T. In some embodiments, the set of modifications comprises V18I, R86T, D87G, T88S, L101Y, K104R, and K105A. In some embodiments, the set of modifications comprises R11I, V18I, R86K, D87G, T88S, L101H, K104R, K105A, and F107M. In some embodiments, the set of modifications comprises L10V, V18I, D87S, D88S, L101F, K104R, K105T, E12A/G/S, and R65D/E. In some embodiments, the set of modifications comprises R11S, V18I, R86K, D87G, T88S, K89M, L101Y, K104R, K105T, E12A/G/S, and R65D/E. In some embodiments, the set of modifications comprises L10V, V18I, D87S, T88S, L101F, K104R, K105T, E12A/G/S, and R65D/E. In some embodiments, the set of modifications comprises L10V/I, D87S, T88S, K89R, L101H/F, K104R, K105T, E12A/G/S, and R65D/E. In some embodiments, the set of modifications comprises L10I, V18I, R86T, D87G, T88S, K89R, L101Y/H, K104R, K105A, E12A/G/S, and R65D/E. In some embodiments, the set of modifications comprises L10V, V18I, D87S, T88S, L101F, K104R, K105T, E12A/G/S, and R65D/E. In some embodiments, the set of modifications comprises V18I, R86T, D87G, T88S, L101Y, K104R, K105A, E12A/G/S, and R65D/E. In some embodiments, the set of modifications comprises R11I, V18I, R86K, D87G, T88S, L101H, K104R, K105A, F107M, E12A/G/S, and R65D/E. In some embodiments, the set of modifications comprises L10V, V18I, D87S, D88S, L101F, K104R, K105T, E12A, and R65D/E. In some embodiments, the set of modifications comprises R11S, V18I, R86K, D87G, T88S, K89M, L101Y, K104R, K105T, E12A, and R65D/E. In some embodiments, the set of modifications comprises L10V, V18I, D87S, T88S, L101F, K104R, K105T, E12A, and R65D/E. In some embodiments, the set of modifications comprises L10V/I, D87S, T88S, K89R, L101H/F, K104R, K105T, E12A, and R65D/E. In some embodiments, the set of modifications comprises L10I, V18I, R86T, D87G, T88S, K89R, L101Y/H, K104R, K105A, E12A, and R65D/E. In some embodiments, the set of modifications comprises L10V, V18I, D87S, T88S, L101F, K104R, K105T, E12A, and R65D/E. In some embodiments, the set of modifications comprises V18I, R86T, D87G, T88S, L101Y, K104R, K105A, E12A, and R65D/E. In some embodiments, the set of modifications comprises R11I, V18I, R86K, D87G, T88S, L101H, K104R, K105A, F107M, E12A, and R65D/E. In some embodiments, the set of modifications comprises L10V, V18I, D87S, D88S, L101F, K104R, K105T, E12G, and R65D/E. In some embodiments, the set of modifications comprises R11S, V18I, R86K, D87G, T88S, K89M, L101Y, K104R, K105T, E12G, and R65D/E. In some embodiments, the set of modifications comprises L10V, V18I, D87S, T88S, L101F, K104R, K105T, E12A/G/S, and R65D/E. In some embodiments, the set of modifications comprises L10V/I, D87S, T88S, K89R, L101H/F, K104R, K105T, E12G, and R65D/E. In some embodiments, the set of modifications comprises L10I, V18I, R86T, D87G, T88S, K89R, L101Y/H, K104R, K105A, E12G, and R65D/E. In some embodiments, the set of modifications comprises L10V, V18I, D87S, T88S, L101F, K104R, K105T, E12G, and R65D/E. In some embodiments, the set of modifications comprises V18I, R86T, D87G, T88S, L101Y, K104R, K105A, E12G, and R65D/E. In some embodiments, the set of modifications comprises R11I, V18I, R86K, D87G, T88S, L101H, K104R, K105A, F107M, E12G, and R65D/E. In some embodiments, the set of modifications comprises L10V, V18I, D87S, D88S, L101F, K104R, K105T, E12S, and R65D/E. In some embodiments, the set of modifications comprises R11S, V18I, R86K, D87G, T88S, K89M, L101Y, K104R, K105T, E12A/G/S, and R65D/E. In some embodiments, the set of modifications comprises L10V, V18I, D87S, T88S, L101F, K104R, K105T, E12S, and R65D/E. In some embodiments, the set of modifications comprises L10V/I, D87S, T88S, K89R, L101H/F, K104R, K105T, E12S, and R65D/E. In some embodiments, the set of modifications comprises L10I, V18I, R86T, D87G, T88S, K89R, L101Y/H, K104R, K105A, E12S, and R65D/E. In some embodiments, the set of modifications comprises L10V, V18I, D87S, T88S, L101F, K104R, K105T, E12S, and R65D/E. In some embodiments, the set of modifications comprises V18I, R86T, D87G, T88S, L101Y, K104R, K105A, E12S, and R65D/E. In some embodiments, the set of modifications comprises R11I, V18I, R86K, D87G, T88S, L101H, K104R, K105A, F107M, E12S, and R65D/E. In some embodiments, the set of modifications comprises L10V, V18I, D87S, D88S, L101F, K104R, K105T, E12A, and R65D. In some embodiments, the set of modifications comprises R11S, V18I, R86K, D87G, T88S, K89M, L101Y, K104R, K105T, E12A, and R65E. In some embodiments, the set of modifications comprises L10V, V18I, D87S, T88S, L101F, K104R, K105T, E12A, and R65D. In some embodiments, the set of modifications comprises L10V/I, D87S, T88S, K89R, L101H/F, K104R, K105T, E12A, and R65D. In some embodiments, the set of modifications comprises L10I, V18I, R86T, D87G, T88S, K89R, L101Y/H, K104R, K105A, E12A, and R65D. In some embodiments, the set of modifications comprises L10V, V18I, D87S, T88S, L101F, K104R, K105T, E12A, and R65D. In some embodiments, the set of modifications comprises V18I, R86T, D87G, T88S, L101Y, K104R, K105A, E12A, and R65D. In some embodiments, the set of modifications comprises R11I, V18I, R86K, D87G, T88S, L101H, K104R, K105A, F107M, E12A, and R65D. In some embodiments, the set of modifications comprises L10V, V18I, D87S, D88S, L101F, K104R, K105T, E12G, and R65D. In some embodiments, the set of modifications comprises R11S, V18I, R86K, D87G, T88S, K89M, L101Y, K104R, K105T, E12G, and R65D. In some embodiments, the set of modifications comprises L10V, V18I, D87S, T88S, L101F, K104R, K105T, E12A/G/S, and R65D. In some embodiments, the set of modifications comprises L10V/I, D87S, T88S, K89R, L101H/F, K104R, K105T, E12G, and R65D. In some embodiments, the set of modifications comprises L10I, V18I, R86T, D87G, T88S, K89R, L101Y/H, K104R, K105A, E12G, and R65D. In some embodiments, the set of modifications comprises L10V, V18I, D87S, T88S, L101F, K104R, K105T, E12G, and R65D. In some embodiments, the set of modifications comprises V18I, R86T, D87G, T88S, L101Y, K104R, K105A, E12G, and R65D. In some embodiments, the set of modifications comprises R11I, V18I, R86K, D87G, T88S, L101H, K104R, K105A, F107M, E12G, and R65D. In some embodiments, the set of modifications comprises L10V, V18I, D87S, D88S, L101F, K104R, K105T, E12S, and R65D. In some embodiments, the set of modifications comprises R11S, V18I, R86K, D87G, T88S, K89M, L101Y, K104R, K105T, E12S, and R65D. In some embodiments, the set of modifications comprises L10V, V18I, D87S, T88S, L101F, K104R, K105T, E12S, and R65D. In some embodiments, the set of modifications comprises L10V/I, D87S, T88S, K89R, L101H/F, K104R, K105T, E12S, and R65D. In some embodiments, the set of modifications comprises L10I, V18I, R86T, D87G, T88S, K89R, L101Y/H, K104R, K105A, E12S, and R65D. In some embodiments, the set of modifications comprises L10V, V18I, D87S, T88S, L101F, K104R, K105T, E12S, and R65D. In some embodiments, the set of modifications comprises V18I, R86T, D87G, T88S, L101Y, K104R, K105A, E12S, and R65D. In some embodiments, the set of modifications comprises R11I, V18I, R86K, D87G, T88S, L101H, K104R, K105A, F107M, E12S, and R65D. In some embodiments, the set of modifications comprises L10V, V18I, D87S, D88S, L101F, K104R, K105T, E12A, and R65E. In some embodiments, the set of modifications comprises R11S, V18I, R86K, D87G, T88S, K89M, L101Y, K104R, K105T, E12A, and R65E. In some embodiments, the set of modifications comprises L10V, V18I, D87S, T88S, L101F, K104R, K105T, E12A, and R65E. In some embodiments, the set of modifications comprises L10V/I, D87S, T88S, K89R, L101H/F, K104R, K105T, E12A, and R65E. In some embodiments, the set of modifications comprises L10I, V18I, R86T, D87G, T88S, K89R, L101Y/H, K104R, K105A, E12A, and R65E. In some embodiments, the set of modifications comprises L10V, V18I, D87S, T88S, L101F, K104R, K105T, E12A, and R65E. In some embodiments, the set of modifications comprises V18I, R86T, D87G, T88S, L101Y, K104R, K105A, E12A, and R65E. In some embodiments, the set of modifications comprises R11I, V18I, R86K, D87G, T88S, L101H, K104R, K105A, F107M, E12A, and R65E. In some embodiments, the set of modifications comprises L10V, V18I, D87S, D88S, L101F, K104R, K105T, E12G, and R65E. In some embodiments, the set of modifications comprises R11S, V18I, R86K, D87G, T88S, K89M, L101Y, K104R, K105T, E12G, and R65E. In some embodiments, the set of modifications comprises L10V, V18I, D87S, T88S, L101F, K104R, K105T, E12A/G/S, and R65E. In some embodiments, the set of modifications comprises L10V/I, D87S, T88S, K89R, L101H/F, K104R, K105T, E12G, and R65E. In some embodiments, the set of modifications comprises L10I, V18I, R86T, D87G, T88S, K89R, L101Y/H, K104R, K105A, E12G, and R65E. In some embodiments, the set of modifications comprises L10V, V18I, D87S, T88S, L101F, K104R, K105T, E12G, and R65E. In some embodiments, the set of modifications comprises V18I, R86T, D87G, T88S, L101Y, K104R, K105A, E12G, and R65E. In some embodiments, the set of modifications comprises R11I, V18I, R86K, D87G, T88S, L101H, K104R, K105A, F107M, E12G, and R65E. In some embodiments, the set of modifications comprises L10V, V18I, D87S, D88S, L101F, K104R, K105T, E12S, and R65E. In some embodiments, the set of modifications comprises R11S, V18I, R86K, D87G, T88S, K89M, L101Y, K104R, K105T, E12A/G/S, and R65E. In some embodiments, the set of modifications comprises L10V, V18I, D87S, T88S, L101F, K104R, K105T, E12S, and R65E. In some embodiments, the set of modifications comprises L10V/I, D87S, T88S, K89R, L101H/F, K104R, K105T, E12S, and R65E. In some embodiments, the set of modifications comprises L10I, V18I, R86T, D87G, T88S, K89R, L101Y/H, K104R, K105A, E12S, and R65E. In some embodiments, the set of modifications comprises L10V, V18I, D87S, T88S, L101F, K104R, K105T, E12S, and R65E. In some embodiments, the set of modifications comprises V18I, R86T, D87G, T88S, L101Y, K104R, K105A, E12S, and R65E. In some embodiments, the set of modifications comprises R11I, V18I, R86K, D87G, T88S, L101H, K104R, K105A, F107M, E12S, and R65E. In some embodiments, the set of modifications comprises L10V, E12A, V18I, R65D, D87S, T88S, L101F, K104R, and K105T (see, for example, IL-13dn; SEQ ID NO:38). In some embodiments, the amino acid seqeunce is 90% identical. In some embodiments, the amino acid sequence is 95% identical. In some embodiments, the amino acid seqeunce is 98% identical. In some embodiments, the amino acid sequence is 99% identical. In some embodiments, the polypeptide comprising the one or more modifications is linked to an an IL-13 superkine immune cell targeting construct. In some embodiments, an IL-13 superkine immune cell targeting construct comprises one or more signaling domains derived from CD3-ζ, CD28, DAP10, OX-40, ICOS and CD137. In some embodiments, an IL-13 superkine immune cell targeting construct comprises one or more signaling domains derived from CD3-ζ. In some embodiments, an IL-13 superkine immune cell targeting construct comprises one or more signaling domains derived from CD28. In some embodiments, an IL-13 superkine immune cell targeting construct comprises one or more signaling domains derived from DAP10. In some embodiments, an IL-13 superkine immune cell targeting construct comprises one or more signaling domains derived from OX-40. In some embodiments, an IL-13 superkine immune cell targeting construct comprises one or more signaling domains derived from CD137. In some embodiments, amino acid substitutions include without limitation those provided in FIG. 2.

Table of IL-13 sequences is provided below.

TABLE 2
List of Amino Acid Sequences

| SEQ ID NO: | |
|---|---|
| (Information) | Amino acid sequence |
| SEQ ID NO: 1 | PGPVPPSTALRELIEELVNITQNQKAPLCNGSMVW |
| (IL-13 | SINLTAGMYCAALESLINVSGCSAIEKTQRMLSGF |
| wildtype) | CPHKVSAGQFSSLHVRDTKIEVAQFVKDLLLHLKK |
| | LFREGQFN |
| SEQ ID NO: 2 | PGPVPPSTAVRALIEELINITQNQKAPLCNGSMVW |
| | SINRTAGMYCAALESLINVSGCSAIEKTQDMLSGF |
| | CPHKVSAGQFSSLHVRSSKIEVAQFVKDLLFHLRT |
| | LFREGQFN |
| SEQ ID NO: 3 | PGPVPPSTAIRELIEELINITQNQKAPLCNGSMVW |
| | SINLTAGMYCAALESLINVSGCSAIEKTQRMLSGF |
| | CPHKVSAGQFSSLHVRGSKIEVAQFVKDLLHHLRA |
| | LFREGQFN |
| SEQ ID NO: 4 | PGPVPPSTAVRELIEELINITQNQKAPLCNGSMVW |
| | SINRTAGMYCAALESLINVSGCSAIEKTQRMLSGF |
| | CPHKVSAGQFSSLHVRSSKIEVAQFVKDLLFHLRT |
| | LFREGQFN |
| SEQ ID NO: 5 | PGPVPPSTALIELIEELINITQNQKAPLCNGSMVW |
| | SINLTAGIYCAALESLINVSGCSAIEKTQRMLSGF |
| | CPHKVSAGQFSSLHVKGSKIEVAQFVKDLLHHLRA |
| | LMREGQFN |
| SEQ ID NO: 6 | PGPVPPSTAIRELIEELLNITQNQKAPLCNGSMVW |
| | SINLTAGMYCAALESLINVSGCSAIEKTQRMLSGF |
| | CPHKVSAGQFSSLHVMKSKIEVAQFVKDLLHHLRA |
| | LFREGQFN |
| SEQ ID NO: 7 | PGPVPPSTAIRELIEELINITQNQKAPLCNGSMVW |
| | SINLTAGMYCAALESLINVSGCSAIEKTQRMLSGF |
| | CPHKVSAGQFSSLHVRSSRIEVAQFVKDLLHHLRT |
| | LFREGQFN |
| SEQ ID NO: 8 | PGPVPPSTALRELIEELINITQNEKAPLCNGSMVW |
| | SINLTAGIYCAALESLINVSGCSAIEKTQRMLSGF |
| | CPHKVSAGQFSSLHVTGSKIEVAQFVKDLLYHLRA |
| | LFREGQFN |
| SEQ ID NO: 9 | PGPVPPSTALSELIEELINITQNQKAPLCNGSMVW |
| | SINPTAGMYCAALESLINVSGCSAIEKTQRMLSGF |
| | CPHKVAAGQFSSLHDKGSMIEVAQFVKDLLYHLRT |
| | LFREGQFN |
| SEQ ID NO: 10 | PGPVPPSTATRELIEELINITQNQKAPLCNGSMVW |
| | SINLTADMYCAALESLINVSGCSAIEKTQRMLSGF |
| | CPHKVSVGQFSSLHVRGSKIEVAQFVKDLLYHLRT |
| | LFREGQFN |
| SEQ ID NO: 11 | PGPVPPSTADIELIAELINITQNQKAPLCNGSMVW |
| | SINLTADMYCAALESLINVSGCSAIEKTQRMLSGF |
| | CPHKVSAGQFSSLHVKKTRIEVAQFVKDLLLHLKK |
| | LFKEGQFN |
| SEQ ID NO: 12 | PGPVPPSTAARELIEELVNITQNQKAPLCNGSMVW |
| | SINLTAGMYCAALESLINVSGCSAIEKTQRMLSGF |
| | CPHKVSAGQLSSLHVTGKRIEVAQFVKDLLNHLRA |
| | LFKEGQFN |
| SEQ ID NO: 13 | PGPVPPSTAVRELIEELVNITQNQKAPLCNGSMVW |
| | SINLTAGMYCAALESLINVSGCSAIEKTQRMLSGF |
| | CPHKVSAGQFSSLHVRDTRIEVAQFVKDLLNHLKE |
| | LFTEGQFN |
| SEQ ID NO: 14 | PGPVPPSTALSELMEELVNITQNQKAPLCNGSMVW |
| | SINLTAGMYCAALESLINVSGCSAIEKTQRMLSGF |
| | CPHKVSAGQFSSLHVRDSKIEVAQFVKDLLNHLKA |
| | LFKEGQFN |
| SEQ ID NO: 15 | GPVPPSTAFRELIEELVNITQNQKAPLCNGSMVWS |
| | INLTAGMYCAALESLINVSGCSAIEKTQRMLSGFC |
| | PHKVSPGQFSSLHVTNSRIEVAQFVKDLLNHLKAL |
| | FKEGQYN |
| SEQ ID NO: 16 | GPVPPSTAHLELIEELINITQNQKAPLCNGSMVWS |
| | INLTAGMYCAALESLINVSGCSAIEKTQRMLSGFC |
| | PHKVSAGQFSSLHVKETRIEVAQFVKDLLNHLKTL |
| | FKEGQFN |
| SEQ ID NO: 17 | PGPVPPSTAHLELIEELINITQNQKAPLCNGSMVW |
| | SINPTAGMYCAALESLINVSGCSAIEKTQRMLSGF |
| | CPHKVSAGQFSSLHVMDTRIEVAQFVKDLLLHLKK |
| | LFKEGQFN |
| SEQ ID NO: 18 | PGPVPPSTAHRELIEELVNITQNQKAPLCNGSMVW |
| | SINLTAGMYCAALESLINVSGCSAIEKTQRMLSGF |
| | CPHKVSAGQFSSLHVTGRKIEVAQFVKDLLLHLKK |
| | LFKEGQFN |
| SEQ ID NO: 19 | PGPVPPSTAHRELIEELVNITQNQKAPLCNGSMVW |
| | RINRTAGMYCAALESLINVSGCSAIEKTQRMLSGF |
| | CPHKVSAGQFSSLHVMDSRIEVAQFVKDLLNHLRA |
| | LFKEGQFN |
| SEQ ID NO: 20 | PGPVPPSTAARELIEELFNITQNQKAPLCNGSMVW |
| | SINLTAGMYCAALESLINVSGCSAIEKTKRMLSGF |
| | CPHKVSAGQFPSLHVKKTRIEVAQFVKDLLIHLRK |
| | LFKEGQFN |
| SEQ ID NO: 21 | PGPVPPSTALIELIEELINITQNQKAPLCNGSMVWS |
| (Exemplary | INLTAGMYCAALESLINVSGCSAIEKTQRMLSGFCP |
| sequence | HKVSAGQFSSLHVKGSKIEVAQFVKDLLHHLRALMR |
| comprising | EGQFN |
| R11I, V18I, | |
| R86K, D87G, | |
| T88S, L101H, | |
| K104R, K105A, | |
| F107M, | |
| referred to | |
| herein as A5) | |
| SEQ ID NO: 22 | PGPVPPSTAIRELIEELLNITQNQKAPLCNGSMVWS |
| (Exemplary | INLTAGMYCAALESLINVSGCSAIEKTQRMLSGFCP |
| sequence | HKVSAGQFSSLHVMKSKIEVAQFVKDLLHHLRALFR |
| comprising | EGQFN |
| L10I, V18L, | |
| R86M, D87K, | |
| T88S, L101H, | |
| K104R, K105A, | |
| referred to | |
| herein as A6) | |
| SEQ ID NO: 23 | PGPVPPSTAIRELIEELINITQNQKAPLCNGSMVWS |
| (Exemplary | INLTAGMYCAALESLINVSGCSAIEKTQRMLSGFCP |
| sequence | HKVSAGQFSSLHVRGSKIEVAQFVKDLLHHLRALFR |
| comprising | EGQFN |
| L10I, V18I, | |
| D87G, T88S, | |
| L101H, K104R, | |
| K105A, | |
| referred to | |
| herein as A7) | |
| SEQ ID NO: 24 | PGPVPPSTAIRELIEELINITQNQKAPLCNGSMVWS |
| (Exemplary | INLTAGMYCAALESLINVSGCSAIEKTQRMLSGFCP |
| sequence | HKVSAGQFSSLHVRSSRIEVAQFVKDLLHHLRTLFR |
| comprising | EGQFN |
| L10I, V18I, | |
| D87S, T88S, | |
| K89R, L101H, | |
| K104R, K105T; | |
| referred to | |
| herein as A8) | |
| SEQ ID NO: 25 | PGPVPPSTAVRELIEELINITQNQKAPLCNGSMVWS |
| (Exemplary | INLTAGMYCAALESLINVSGCSAIEKTQRMLSGFCP |
| sequence | HKVSAGQFSSLHVRSSKIEVAQFVKDLLFHLRTLFR |
| comprising | EGQFN |
| L10V, V18I, | |
| D87S, T88S, | |
| L101F, K104R, | |
| K105T, | |
| referred to | |
| herein as | |
| A11) | |
| SEQ ID NO: 26 | PGPVPPSTALRELIEELINITQNQKAPLCNGSMVW |
| (Exemplary | SINLTAGMYCAALESLINVSGCSAIEKTQRMLSGF |
| sequence | CPHKVSAGQFSSLHVTGSKIEVAQFVKDLLYHLRA |
| comprising | LFREGQFN |
| V18I, R86T, | |
| D87G, T88S, | |
| L101Y, K104R, | |
| K105A, | |
| referred to | |
| herein as B2) | |
| SEQ ID NO: 27 | PGPVPPSTALSELIEELINITQNQKAPLCNGSMVW |
| (Exemplary | SINLTAGMYCAALESLINVSGCSAIEKTQRMLSGF |
| sequence | CPHKVSAGQFSSLHVKGSMIEVAQFVKDLLYHLRT |
| comprising | LFREGQFN |
| R11S, V18I, | |
| R86K, D87G, | |
| T88S, K89M, | |
| L101Y, K104R, | |
| K105T, | |
| referred to | |
| herein as B4) | |
| SEQ ID NO: 28 | PGPVPPSTATRELIEELINITQNQKAPLCNGSMVW |
| (Exemplary | SINLTAGMYCAALESLINVSGCSAIEKTQRMLSGF |
| sequence | CPHKVSAGQFSSLHVRGSKIEVAQFVKDLLYHLRT |
| comprising | LFREGQFN |
| L10T, V18I, | |
| D87G, T88S, | |
| K89K, L10Y1, | |
| K104R, K105T, | |
| referred to | |
| herein as B6) | |
| SEQ ID NO: 29 | PGPVPPSTADIELIEELINITQNQKAPLCNGSMVW |
| (Exemplary | SINLTAGMYCAALESLINVSGCSAIEKTQRMLSGF |
| sequence | CPHKVSAGQFSSLHVKKTRIEVAQFVKDLLLHLKK |
| comprising | LFKEGQFN |
| L10D, R11I, | |
| V18I, R86K, | |
| D87K, K89R, | |
| R108K, | |
| referred to | |
| herein as C2) | |
| SEQ ID NO: 30 | PGPVPPSTAARELIEELVNITQNQKAPLCNGSMVW |
| (Exemplary | SINLTAGMYCAALESLINVSGCSAIEKTQRMLSGF |
| sequence | CPHKVSAGQFSSLHVTGKRIEVAQFVKDLLNHLRA |
| comprising | LFKEGQFN |
| L10A, R86T, | |
| D87G, T88K, | |
| K89R, L101N, | |
| K104R, K105A, | |
| R108K, | |
| referred to | |
| herein as C3) | |
| SEQ ID NO: 31 | PGPVPPSTAVRELIEELVNITQNQKAPLCNGSMVW |
| (Exemplary | SINLTAGMYCAALESLINVSGCSAIEKTQRMLSGF |
| sequence | CPHKVSAGQFSSLHVRDTRIEVAQFVKDLLNHLKE |
| comprising | LFTEGQFN |
| L10V, K89R, | |
| L101N, K105E, | |
| R108T, | |
| referred to | |
| herein as C4) | |
| SEQ ID NO: 32 | PGPVPPSTALSELMEELVNITQNQKAPLCNGSMVW |
| (Exemplary | SINLTAGMYCAALESLINVSGCSAIEKTQRMLSGF |
| sequence | CPHKVSAGQFSSLHVRDSKIEVAQFVKDLLNHLKA |
| comprising | LFKEGQFN |
| R11S, I14M, | |
| T88S, L101N, | |
| K105A, R108K, | |
| referred to | |
| herein as C7) | |
| SEQ ID NO: 33 | PGPVPPSTAHLELIEELINITQNQKAPLCNGSMVW |
| (Exemplary | SINLTAGMYCAALESLINVSGCSAIEKTQRMLSGF |
| sequence | CPHKVSAGQFSSLHVKETRIEVAQFVKDLLNHLKT |
| comprising | LFKEGQFN |
| L10H, R11L, | |
| V18I, R86K, | |
| D87E, K89R, | |
| L101N, K105T, | |
| R108K, | |
| refered to | |
| herein as C9) | |
| SEQ ID NO: 34 | PGPVPPSTAHLELIEELINITQNQKAPLCNGSMVW |
| (Exemplary | SINLTAGMYCAALESLINVSGCSAIEKTQRMLSGF |
| sequence | CPHKVSAGQFSSLHVMDTRIEVAQFVKDLLLHLKK |
| comprising | LFKEGQFN |
| L10H, R11L, | |
| V18I, R86M, | |
| K89R, R108K, | |
| referred to | |
| herein as | |
| C10) | |
| SEQ ID NO: 35 | PGPVPPSTAHRELIEELVNITQNQKAPLCNGSMVW |
| (Exemplary | SINLTAGMYCAALESLINVSGCSAIEKTQRMLSGF |
| sequence | CPHKVSAGQFSSLHVTGRKIEVAQFVKDLLLHLKK |
| comprising | LFKEGQFN |
| L10H, R86T, | |
| D87G, T88R, | |
| R108K, | |
| referred to | |
| herein as | |
| C11) | |
| SEQ ID NO: 36 | PGPVPPSTAHRELIEELVNITQNQKAPLCNGSMVW |
| (Exemplary | SINLTAGMYCAALESLINVSGCSAIEKTQRMLSGF |
| sequence | CPHKVSAGQFSSLHVMDSRIEVAQFVKDLLNHLRA |
| comprising | LFKEGQFN |
| L10H, R86M, | |
| T88S, K89R, | |
| L101N, K104R, | |
| K105A, R108K, | |
| referred to | |
| herein as | |
| C12) | |
| SEQ ID NO: 37 | PGPVPPSTAARELIEELFNITQNQKAPLCNGSMVW |
| (Exemplary | SINLTAGMYCAALESLINVSGCSAIEKTQRMLSGF |
| sequence | CPHKVSAGQFSSLHVKKTRIEVAQFVKDLLIHLRK |
| comprising | LFKEGQFN |
| L10A, V18F, | |
| R86F, D87F, | |
| K89R, L101I, | |
| K104R, R108K, | |
| referred to | |
| herein as D7) | |
| SEQ ID NO: 38 | PGPVPPSTAVRALIEELINITQNQKAPLCNGSMVW |
| (Exemplary | SINLTAGMYCAALESLINVSGCSAIEKTQDMLSGF |
| sequence | CPHKVSAGQFSSLHVRSSKIEVAQFVKDLLFHLRT |
| comprising | LFREGQFN |
| L10V, E12A, | |
| V18I, R65D, | |
| D87S, T88S, | |
| L101F, K104R, | |
| K105T, | |
| referred to | |
| herein as | |
| IL-13dn) | |

Included as superkines are amino acid and nucleic acid coding sequences that are 90%, 95%, 98% or 99% identical to these sequences, longer sequences that comprise those sequences but also include additional nucleotides at the 3′ or 5′ end, for example any number of additional nucleotides or codons, such as 3, 6, 9, 12 or more nucleotides, or up to about 12, 20, 50 or 100 additional nucleotides, and any sequence that encodes the same amino acid sequence as these nucleic acids due to the degeneracy of the genetic code. In particular, sequences that are codon optimized (CO) for expression by the desired host are contemplated as part of the invention. In some embodiments, the amino acid seqeunce is 90% identical. In some embodiments, the amino acid sequence is 95% identical. In some embodiments, the amino acid seqeunce is 98% identical. In some embodiments, the amino acid sequence is 99% identical. In some embodiments, the polypeptide is linked to an an IL-13 superkine immune cell targeting construct. In some embodiments, an IL-13 superkine immune cell targeting construct comprises one or more signaling domains derived from CD3-ζ, CD28, DAP10, OX-40, ICOS and CD137. In some embodiments, an IL-13 superkine immune cell targeting construct comprises one or more signaling domains derived from CD3-ζ. In some embodiments, an IL-13 superkine immune cell targeting construct comprises one or more signaling domains derived from CD28. In some embodiments, an IL-13 superkine immune cell targeting construct comprises one or more signaling domains derived from DAP10. In some embodiments, an IL-13 superkine immune cell targeting construct comprises one or more signaling domains derived from OX-40. In some embodiments, an IL-13 superkine immune cell targeting construct comprises one or more signaling domains derived from CD137. In some embodiments, an IL-13 superkine immune cell targeting construct comprises an IL-13 variant/IL-13 superkine including those provided in FIG. 2. In some embodiments, an IL-13 superkine immune cell targeting construct comprises an IL-13 variant/IL-13 superkine including those provided in SEQ ID NO:2 through SEQ ID NO:38.

### NK Cells

In some embodiments the immune cells are natural killer (NK) cells. NK cells recognize infected or transformed cells through multiple cell surface receptors including NKG2D, CD16, and natural cytotoxicity receptors (NCRs) such as NKp44, NKp46, and NKp30. These receptors activate signaling adapter proteins such as DAP10, DAP12, and CD3ζ, which contain immuno-tyrosine activation motifs (ITAMs) that initiate the release of cytolytic granules containing perforin and granzymes, as well as mediate production and release of cytokines and chemokines such as IFN-γ and TNF-α. Importantly, NK cell-mediated cytotoxicity does not rely on the presentation of self HLA. Therefore, NK cells hold significant clinical interest as a cell-based therapy for cancer because of their ability to be used in an allogeneic setting and potentially provide an off-the-shelf cellular product.

Natural killer cells provide an alternative to the use of T cells for adoptive immunotherapy since they do not require HLA matching, so can be used as allogeneic effector cells. Clinical trials of adoptively transferred allogeneic NK cells demonstrate these cells can survive in patients for several weeks to months. Additionally, expression of CARs in NK cells allow these cells to more effectively kill solid tumors that are often resistant to NK cell-mediated activity compared to hematologic malignancies (especially acute myelogenous leukemia) that are typically more NK cell-sensitive. CARs useful in NK cell targeting include, for example, first generation CAR constructs that contain CD3ζ as the sole signaling domain. Second and third generation CARs are also useful in NK cells. In some embodiments the ectodomain of NKG2D, an NK cell activation receptor, is linked directly to CD3ζ.

NK cells for modification include cell lines, or peripheral blood NK cells, which can be isolated from donors through simple blood draws or by apheresis if larger numbers of cells are needed. Activated PB-NK cells express a wider range of activating receptors, such as CD16, NKp44, and NKp46 as well as KIRs, which play an important role in NK cell licensing. In addition, PB-NK cells can be given without irradiating the cells so have the ability to expand in vivo. Another source of NK cells suitable for CAR expression are NK cells derived from human pluripotent stem cells—both induced pluripotent stem cells (iPSCs) or human embryonic stem cells (hESCs). These NK cells display a similar phenotype to PB-NK cells, and hESC/iPSC-NK cells can be grown on a clinical scale.

### CARs

In addition to the superkine sequence, CARs contain the signaling domain for CD3ζ and the signaling domains of one or more costimulatory receptors that further promote the recycling, survival and/or expansion of immune cells expressing the CARs. The signaling domains of the costimulatory receptors are the intracellular portions of each receptor protein that generate the activating signal in the cell. Examples are amino acids 180-220 of the native CD28 molecule and amino acids 214-255 of the native 4-1BB molecule.

Examples of suitable hinge and transmembrane regions to link the superkine to the signaling region may include without limitation the constant (Fc) regions of immunoglobins, human CD8a, and artificial linkers that serve to move the targeting moiety away from the cell surface for improved access to and binding on target cells. Examples of suitable transmembrane domains include the transmembrane domains of the leukocyte CD markers, preferably that of CD4 or CD28. Examples of intracellular receptor signaling domains include the T cell antigen receptor complex, preferably the zeta chain of CD3, however any transmembrane region sufficient to anchor the CAR in the membrane can be used. Persons of skill are aware of numerous transmembrane regions and the structural elements (such as lipophilic amino acid regions) that produce transmembrane domains in numerous membrane proteins and therefore can substitute any convenient sequence. T cell costimulatory signaling receptors suitable for improving the function and activity of CAR-expressing cells include, but are not limited to, CD28, CD137, and OX-40.

Signaling via CD28 is required for IL2 production and proliferation, but does not play a primary role in sustaining T cell function and activity. CD137 (a tumor necrosis factor-receptor family member expressed following CD28 activation) and OX-40 are involved in driving long-term survival of T cells, and accumulation of T cells. The ligands for these receptors typically are expressed on professional antigen presenting cells such as dendritic cells and activated macrophages, but not on tumor cells. Expressing a CAR that incorporates CD28 and/or 4-1BB signaling domains in CD4⁺ T cells enhances the activity and anti-tumor potency of those cells compared to those expressing a CAR that contains only the CD3ζ signaling domain, which constructs may be referred to as second or third generation CARs.

Included as CAR constructs of interest are tandem CARs, e.g. see Hegde et al. (2016) J. Clin. Invest 126(8):3036-3052, herein specifically incorporated by reference. In such constructs a binding moiety for a tumor specific antigen is combined in tandem with an IL-13 superkine. The binding moiety may be, for example, an scFv specific for a tumor cell antigen, including without limitation HER-2, EGFR, CD20, etc. as known in the art.

### BiTES

Bi-specific T-cell engagers (BiTEs) are fusion proteins comprising an IL-13 superkine fused to an antibody variable region that specifically binds to CD3. In some embodiments the antibody variable region in a single-chain variable fragments (scFvs). THe superkine may be fused to the variable region through a linker. An Fc region is optionally provided.

### TACs

A TAC construct comprises an IL-13 superkine fused to a ligand that binds a protein associated with the TCR complex; fused to a T cell receptor signaling domain polypeptide. The domains may be separated by linkers. The protein associated with the TCR complex may be CD3. The ligand that binds a protein associated with the TCR complex may be a single chain antibody. The ligand that binds a protein associated with the TCR complex may be UCHT1, or a variant thereof. The T cell receptor signaling domain polypeptide may comprise a cytosolic domain and a transmembrane domain. The cytosolic domain may be a CD4 cytosolic domain and the transmembrane domain is a CD4 transmembrane domain.

### ACTRs

ACTRs are a hybrid approach to CARs and the established monoclonal antibody oncology therapeutics. ACTRs are composed of a typical CAR construct that can bind the heavy chain of an antibody through a high-affinity variant of the Fc receptor CD16. A superkine is fused to a moiety recognized by the CAR, which may include, without limitation, an Fc region of an antibody with high affinity for CD16.

An immune cell targeting construct coding sequence can be produced by any means known in the art, including recombinant DNA techniques. Nucleic acids encoding the several regions of the chimeric receptor can be prepared and assembled into a complete coding sequence by standard techniques of molecular cloning known in the art (genomic library screening, PCR, primer-assisted ligation, site-directed mutagenesis, etc.) as is convenient. The resulting coding region may be inserted into an expression vector and used to transform a suitable expression host cell line, e.g. a population of allogeneic or autologous T lymphocytes, allogeneic or autologous NK cells, including primary cultures, cell lines, iPSC derived cells, etc. The methods can be used on cells in vitro (e.g., in a cell-free system), in culture, e.g. in vitro or ex vivo. For example, IL-13 superkine CAR-expressing cells can be cultured and expanded in vitro in culture medium.

An IL13 superkine immune cell targeting construct can specifically direct immune cells to target IL13Rα2-expressing glioma cells, renal carcinoma cells and cells of any cancer expressing IL13Rα2, in an MHC-independent manner. IL13Rα2 has been identified as an over-expressed cell-surface target on various human tumors, including breast cancer, head and neck cancer, kidney cancer, ovarian cancer and Kaposi's sarcoma as well as gliomas. Anti-tumor effector cells, e.g. CD4⁺ or CD8⁺ effector T cells, are generated to be re-directed to recognize such tumor cells by introducing into the T cells an IL-13 superkine immune cell targeting construct comprising one or more signaling domains derived from CD3-ζ, CD28, DAP10, OX-40, ICOS and CD137.

The IL-13 superkine immune cell targeting construct is infected or transfected into human immune cells, e.g. using a non-viral plasmid vector and electroporation methods; a viral vector and infection methods, etc. as known in the art. A CAR comprising co-stimulatory signaling domains may enhance the duration and/or retention of anti-tumor activity in a manner that can significantly improve the clinical efficacy of adoptive therapy protocols. CD4⁺ and CD8⁺ T cell effector functions, and NK cell functions can be triggered via these receptors, therefore these cell types are contemplated for use with the invention. CD8⁺ T cells expressing the IL13 superkine CARs of this invention may be used to lyse target cells and to produce IL-2 in the presence of target cells, among the other functions of these cells. Expression of the appropriate costimulatory CAR in either or both CD4⁺ and CD8⁺ T cells is used to provide the most effective population of cells for adoptive immunotherapy, consisting therefore of either or both professional helper and killer T cells that exhibit enhanced and/or long term viability and anti-tumor activity. In some embodiments, an IL-13 superkine immune cell targeting construct comprises an IL-13 variant/IL-13 superkine including those provided in FIG. 2. In some embodiments, an IL-13 superkine immune cell targeting construct comprises an IL-13 variant/IL-13 superkine including those provided in SEQ ID NO:2 through SEQ ID NO:38.

Polypeptides of the present invention can be further modified, e.g., joined to a wide variety of other oligopeptides or proteins for a variety of purposes. For example, post-translationally modified, for example by prenylation, acetylation, amidation, carboxylation, glycosylation, pegylation, etc. Such modifications can also include modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes.

Methods which are well known to those skilled in the art can be used to construct T cell targeting construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized. One of skill in the art can readily utilize well-known codon usage tables and synthetic methods to provide a suitable coding sequence for any of the polypeptides of the invention. The nucleic acids may be isolated and obtained in substantial purity. Usually, the nucleic acids, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome. The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art.

According to the present invention, immune cell targeting construct vectors and immune cell targeting construct modified cells can be provided in pharmaceutical compositions suitable for therapeutic use, e.g. for human treatment. In some embodiments, pharmaceutical compositions of the present invention include one or more therapeutic entities of the present invention or pharmaceutically acceptable salts, esters or solvates thereof. In some other embodiments, pharmaceutical compositions of the present invention include one or more therapeutic entities of the present invention in combination with another therapeutic agent, e.g., another anti-tumor agent.

Therapeutic entities of the present invention are often administered as pharmaceutical compositions comprising an active therapeutic agent and a other pharmaceutically acceptable excipient. Such formulations can include one or more non-toxic pharmaceutically acceptable carriers, diluents, excipients and/or adjuvants. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions of the present invention can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

The maximum tolerated dose (MTD) of CAR immune cells may be determined during clinical trial development, for example at up to about 10⁴ T cells/kg of body weight, up to about 10⁵ cells/kg of body weight, up to about 10⁶ cells/kg of body weight, up to about 5×10⁶ cells/kg of body weight, up to about 10⁷ cells/kg of body weight, up to about 5×10⁷ cells/kg of body weight, or more, as empirically determined. In some embodiments, the maximum tolerated dose (MTD) of CAR immune cells is up to about 10⁴ T cells/kg of body weight. In some embodiments, the maximum tolerated dose (MTD) of CAR immune cells is up to about 10⁵ T cells/kg of body weight. In some embodiments, the maximum tolerated dose (MTD) of CAR immune cells is up to about 10⁶ T cells/kg of body weight. In some embodiments, the maximum tolerated dose (MTD) of CAR immune cells is up to about 10⁷ T cells/kg of body weight. In some embodiments, the maximum tolerated dose (MTD) of CAR immune cells is up to about 5×10⁶ T cells/kg of body weight. In some embodiments, the maximum tolerated dose (MTD) of CAR immune cells is up to about 5×10⁷ T cells/kg of body weight.

Toxicity of the cells described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

After a dose escalation phase, patients in the expansion cohort are treated with immune cells at the MTD. An exemplary treatment regime entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient.

In prophylactic applications, e.g. to maintain remission in a patient, a relatively low dosage may be administered at relatively infrequent intervals over a long period of time. Some patients continue to receive treatment for the rest of their lives. In other therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Examples of additional therapeutic agents that can be coadministered and/or coformulated with an immune cell targeting construct include: anti-proliferative, or cytoreductive therapy, which is used therapeutically to eliminate tumor cells and other undesirable cells in a host, and includes the use of therapies such as delivery of ionizing radiation, and administration of chemotherapeutic agents. Chemotherapeutic agents are well-known in the art and are used at conventional doses and regimens, or at reduced dosages or regimens, including for example, topoisomerase inhibitors such as anthracyclines, including the compounds daunorubicin, adriamycin (doxorubicin), epirubicin, idarubicin, anamycin, MEN 10755, and the like. Other topoisomerase inhibitors include the podophyllotoxin analogues etoposide and teniposide, and the anthracenediones, mitoxantrone and amsacrine. Other anti-proliferative agent interferes with microtubule assembly, e.g. the family of vinca alkaloids. Examples of vinca alkaloids include vinblastine, vincristine; vinorelbine (NAVELBINE); vindesine; vindoline; vincamine; etc. DNA-damaging agent include nucleotide analogs, alkylating agents, etc. Alkylating agents include nitrogen mustards, e.g. mechlorethamine, cyclophosphamide, melphalan (L-sarcolysin), etc.; and nitrosoureas, e.g. carmustine (BCNU), lomustine (CCNU), semustine (methyl-CCNU), streptozocin, chlorozotocin, etc. Nucleotide analogs include pyrimidines, e.g. cytarabine (CYTOSAR-U), cytosine arabinoside, fluorouracil (5-FU), floxuridine (FUdR), etc.; purines, e.g. thioguanine (6-thioguanine), mercaptopurine (6-MP), pentostatin, fluorouracil (5-FU) etc.; and folic acid analogs, e.g. methotrexate, 10-propargyl-5,8-dideazafolate (PDDF, CB3717), 5,8-dideazatetrahydrofolic acid (DDATHF), leucovorin, etc. Other chemotherapeutic agents of interest include metal complexes, e.g. cisplatin (cis-DDP), carboplatin, oxaliplatin, etc.; ureas, e.g. hydroxyurea; and hydrazines, e.g. N-methylhydrazine.

For example, ionizing radiation (IR) is used to treat about 60% of cancer patients, by depositing energy that injures or destroys cells in the area being treated, and for the purposes of the present invention may be delivered at conventional doses and regimens, or at reduced doses. Radiation injury to cells is nonspecific, with complex effects on DNA. The efficacy of therapy depends on cellular injury to cancer cells being greater than to normal cells. Radiotherapy may be used to treat every type of cancer. Some types of radiation therapy involve photons, such as X-rays or gamma rays. Another technique for delivering radiation to cancer cells is internal radiotherapy, which places radioactive implants directly in a tumor or body cavity so that the radiation dose is concentrated in a small area. A suitable dose of ionizing radiation may range from at least about 2 Gy to not more than about 10 Gy, usually about 5 Gy. A suitable dose of ultraviolet radiation may range from at least about 5 J/m² to not more than about 50 J/m², usually about 10 J/m². The sample may be collected from at least about 4 and not more than about 72 hours following ultraviolet radiation, usually around about 4 hours.

Treatment may also be combined with immunoregulatory modulating agents, including an agent that agonizes an immune costimulatory molecule, e.g. CD40, OX40, etc.; and/or (iii) an agent that antagonizes an immune inhibitory molecule, e.g. CTLA-4, PD-1, PD-L1, etc. The active agents are administered within a period of time to produce an additive or synergistic effect on depletion of cancer cells in the host. Methods of administration include, without limitation, systemic administration, intra-tumoral administration, etc.

In some embodiments, an individual cancer is selected for treatment with a combination therapy because the cancer is a cancer type that is responsive to a checkpoint inhibitor, e.g. a PD-1 antagonist, a PD-L1 antagonist, a CTLA4 antagonist, a TIM-3 antagonist, a BTLA antagonist, a VISTA antagonist, a LAG3 antagonist; etc. In some embodiments, such an immunoregulatory agent is a CTLA-4, PD1 or PDL1 antagonist, e.g. avelumab, nivolumab, pembrolizumab, ipilimumab, and the like. In some such embodiments the cancer is, without limitation, melanoma or small cell lung cancer. In some such embodiments, the cancer is a type that has a high neoantigen, or mutagenesis, burden (see Vogelstein et al. (2013) Science 339(6127):1546-1558, herein specifically incorporated by reference).

In some embodiments, an individual cancer is selected for treatment with a combination therapy of the present invention because the cancer is a cancer type that is responsive to an immune response agonist, e.g. a CD28 agonist, an OX40 agonist; a GITR agonist, a CD137 agonist, a CD27 agonist, an HVEM agonist, etc. In some embodiments, such an immunoregulatory agent is an OX40, CD137, or GITR agonist e.g. tremelimumab, and the like. In some such embodiments the cancer is, without limitation, melanoma or small cell lung cancer. In some such embodiments, the cancer is a type that has a high neoantigen, or mutagenesis, burden.

In some embodiments, the combination therapy includes an antibody known in the art which binds to PD-1 and disrupt the interaction between the PD-1 and its ligand, PD-L1, and stimulate an anti-tumor immune response. In some embodiments, the antibody or antigen-binding portion thereof binds specifically to PD-1. For example, antibodies that target PD-1 and which can find used in the present invention include, e.g., but are not limited to nivolumab (BMS-936558, Bristol-Myers Squibb), pembrolizumab (lambrolizumab, MK03475 or MK-3475, Merck), humanized anti-PD-1 antibody JS001 (ShangHai JunShi), monoclonal anti-PD-1 antibody TSR-042 (Tesaro, Inc.), Pidilizumab (anti-PD-1 mAb CT-011, Medivation), anti-PD-1 monoclonal Antibody BGB-A317 (BeiGene), and/or anti-PD-1 antibody SHR-1210 (ShangHai HengRui), human monoclonal antibody REGN2810 (Regeneron), human monoclonal antibody MDX-1106 (Bristol-Myers Squibb), and/or humanized anti-PD-1 IgG4 antibody PDR001 (Novartis). In some embodiments, the PD-1 antibody is from clone: RMP1-14 (rat IgG)—BioXcell cat #BP0146. Other suitable antibodies include anti-PD-1 antibodies disclosed in U.S. Pat. No. 8,008,449, herein incorporated by reference. In some embodiments, the antibody or antigen-binding portion thereof binds specifically to PD-L1 and inhibits its interaction with PD-1, thereby increasing immune activity. Any antibodies known in the art which bind to PD-L1 and disrupt the interaction between the PD-1 and PD-L1, and stimulates an anti-tumor immune response, are suitable for use in the combination treatment methods disclosed herein. For example, antibodies that target PD-L1 and are in clinical trials, include BMS-936559 (Bristol-Myers Squibb) and MPDL3280A (Genetech). Other suitable antibodies that target PD-LI are disclosed in U.S. Pat. No. 7,943,743, herein incorporated by reference. It will be understood by one of ordinary skill that any antibody which binds to PD-1 or PD-L1, disrupts the PD-1/PD-L1 interaction, and stimulates an anti-tumor immune response, is suitable for use in the combination treatment methods.

In some embodiments, the combination therapy includes an antibody known in the art which binds CTLA-4 and disrupts its interaction with CD80 and CD86. Exemplary antibodies that target CTLA-4 include ipilimumab (MDX-010, MDX-101, Bristol-Myers Squibb), which is FDA approved, and tremelimumab (ticilimumab, CP-675, 206, Pfizer), currently undergoing human trials. Other suitable antibodies that target CTLA-4 are disclosed in WO 2012/120125, U.S. Pat. Nos. 6,984,720, 6,682,7368, and U.S. Patent Applications 2002/0039581, 2002/0086014, and 2005/0201994, herein incorporated by reference. It will be understood by one of ordinary skill that any antibody which binds to CTLA-4, disrupts its interaction with CD80 and CD86, and stimulates an anti-tumor immune response, is suitable for use in the combination treatment methods. In some embodiments, the combination therapy includes an antibody known in the art which binds LAG-3 and disrupts its interaction with MHC class II molecules. An exemplary antibody that targets LAG-3 is IMP321 (Immutep), currently undergoing human trials. Other suitable antibodies that target LAG-3 are disclosed in U.S. Patent Application 2011/0150892, herein incorporated by reference. It will be understood by one of ordinary skill that any antibody which binds to LAG-3, disrupts its interaction with MHC class II molecules, and stimulates an anti-tumor immune response, is suitable for use in the combination treatment methods.

In some embodiments, the combination therapy includes an antibody known in the art which binds TIM-3 and disrupts its interaction with galectin 9. Suitable antibodies that target TIM-3 are disclosed in U.S. Patent Application 2013/0022623, herein incorporated by reference. It will be understood by one of ordinary skill that any antibody which binds to TIM-3, disrupts its interaction with galectin 9, and stimulates an anti-tumor immune response, is suitable for use in the combination treatment methods.

In some embodiments, the combination therapy includes an antibody known in the art which binds 4-1BB/CD137 and disrupts its interaction with CD137L. It will be understood by one of ordinary skill that any antibody which binds to 4-1BB/CD137, disrupts its interaction with CD137L or another ligand, and stimulates an anti-tumor immune response or an immune stimulatory response that results in anti-tumor activity overall, is suitable for use in the combination treatment methods.

In some embodiments, the combination therapy includes an antibody known in the art which binds GITR and disrupts its interaction with its ligand. It will be understood by one of ordinary skill that any antibody which binds to GITR, disrupts its interaction with GITRL or another ligand, and stimulates an anti-tumor immune response or an immune stimulatory response that results in anti-tumor activity overall, is suitable for use in the combination treatment methods.

In some embodiments, the combination therapy includes an antibody known in the art which binds OX40 and disrupts its interaction with its ligand. It will be understood by one of ordinary skill that any antibody which binds to OX40, disrupts its interaction with OX40L or another ligand, and stimulates an anti-tumor immune response or an immune stimulatory response that results in anti-tumor activity overall, is suitable for use in the combination treatment methods.

In some embodiments, the combination therapy includes an antibody known in the art which binds CD40 and disrupts its interaction with its ligand. It will be understood by one of ordinary skill that any antibody which binds to CD40, disrupts its interaction with its ligand, and stimulates an anti-tumor immune response or an immune stimulatory response that results in anti-tumor activity overall, is suitable for use in the combination treatment methods.

In some embodiments, the combination therapy includes an antibody known in the art which binds ICOS and disrupts its interaction with its ligand. It will be understood by one of ordinary skill that any antibody which binds to ICOS, disrupts its interaction with its ligand, and stimulates an anti-tumor immune response or an immune stimulatory response that results in anti-tumor activity overall, is suitable for use in the combination treatment methods.

In some embodiments, the combination therapy includes an antibody known in the art which binds CD28 and disrupts its interaction with its ligand. It will be understood by one of ordinary skill that any antibody which binds to CD28, disrupts its interaction with its ligand, and stimulates an anti-tumor immune response or an immune stimulatory response that results in anti-tumor activity overall, is suitable for use in the combination treatment methods.

In some embodiments, the combination therapy includes an antibody known in the art which binds IFNα and disrupts its interaction with its ligand. It will be understood by one of ordinary skill that any antibody which binds to IFNα, disrupts its interaction with its ligand, and stimulates an anti-tumor immune response or an immune stimulatory response that results in anti-tumor activity overall, is suitable for use in the combination treatment methods.

An “anti-cancer therapeutic” is a compound, composition, or treatment (e.g., surgery) that prevents or delays the growth and/or metastasis of cancer cells. Such anti-cancer therapeutics include, but are not limited to, surgery (e.g., removal of all or part of a tumor), chemotherapeutic drug treatment, radiation, gene therapy, hormonal manipulation, immunotherapy (e.g., therapeutic antibodies and cancer vaccines) and antisense or RNAi oligonucleotide therapy. Examples of useful chemotherapeutic drugs include, but are not limited to, hydroxyurea, busulphan, cisplatin, carboplatin, chlorambucil, melphalan, cyclophosphamide, Ifosphamide, danorubicin, doxorubicin, epirubicin, mitoxantrone, vincristine, vinblastine, Navelbine® (vinorelbine), etoposide, teniposide, paclitaxel, docetaxel, gemcitabine, cytosine, arabinoside, bleomycin, neocarcinostatin, suramin, taxol, mitomycin C, Avastin, Herceptin®, flurouracil, and temozolamide and the like. The compounds are also suitable for use with standard combination therapies employing two or more chemotherapeutic agents. It is to be understood that anti-cancer therapeutics includes novel compounds or treatments developed in the future.

The pharmaceutical compositions and/or formulations described above include one or more therapeutic entities in an amount effective to achieve the intended purpose. Thus the term “therapeutically effective dose” refers to the amount of the therapeutic entities that ameliorates the symptoms of cancer. Determination of a therapeutically effective dose of a compound is well within the capability of those skilled in the art. For example, the therapeutically effective dose can be estimated initially either in cell culture assays, or in animal models, such as those described herein. Animal models can also be used to determine the appropriate concentration range and route of administration. Such information can then be used to determine useful doses and routes for administration in other animals, including humans, using standard methods known in those of ordinary skill in the art.

Also within the scope of the invention are kits comprising the compositions of the invention and instructions for use. The kit may further contain a least one additional reagent, e.g. a chemotherapeutic drug, anti-tumor antibody, etc. Kits typically include a label indicating the intended use of the contents of the kit. The term label includes any writing, or recorded material supplied on or with the kit, or which otherwise accompanies the kit.

The invention now being fully described, it will be apparent to one of ordinary skill in the art that various changes and modifications can be made without departing from the spirit or scope of the invention. In some embodiments, the kit comprises an IL-13 superkine immune cell targeting construct comprising an IL-13 variant/IL-13 superkine as described herein. In some embodiments, the kit comprises an IL-13 superkine immune cell targeting construct comprising an IL-13 variant/IL-13 superkine including those provided in FIG. 2. In some embodiments, an IL-13 superkine immune cell targeting construct comprises an IL-13 variant/IL-13 superkine including those provided in SEQ ID NO:2 through SEQ ID NO:38.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Methods

Protein expression and purification. Human IL-13 and human IL-13Rα1 and IL-13Rα2-selective variants were cloned into the insect expression vector pAcGP67 (BD Biosciences) with C-terminal 6×Histidine tag and produced in insect Hi5 cells using recombinant baculovirus. Proteins were recovered from Hi5 supernatant after 60 hr of infection by nickel agarose and concentrated and purified by size exclusion chromatography on a Superdex-200 column into HBS (10 mM Hepes pH 7.4, 150 mM NaCl). biotinylated IL-13Rα1 (amino acids 1-310) and IL-4Rα1 (amino acids 1-202) ectodomains were obtained by cloning into the pAcGP67-A vector with a C-terminal biotin acceptor peptide (BAP)-LNDIFEAQKIEWHE and hexahistidine tag. Receptor proteins were coexpressed with BirA ligase with excess biotin (100 μM).

Surface Plasmon Resonance. SPR experiments were conducted on a Biacore T100 instrument. Experiments used a Biacore SA sensor chip (GE Healthcare). Biotinylated IL-13Rα1 and IL-13Rα2 receptors were captured at a low density (100-200 RU) and kinetic runs were conducted at 40 μL/min. An unrelated biotinylated protein was immobilized as a reference surface for the SA sensor chip with matching RU to the experimental surface. All data was analyzed using the Biacore T100 evaluation software version 2.0 with a 1:1 Langmuir binding model. Serial dilutions of unbiotinylated IL-13 variants in the running buffer [1×HBS-P (GE Healthcare)+0.5% BSA] were flowed over the chip and IL-13Rα1/IL-13Rα2 were regenerated by using one 60 second injections of 7 mM glycine (pH 3.0).

Phospho-flow cytometry assay. The IL-13 responsive cell line A549 was stimulated with the indicated doses of IL-13 and IL-13 specific variants for 15 min. Samples were then fixed in PFA for 15 min at room temperature, washed with PBS 0.5% BSA and permeabilized with cold (4° C.) methanol for 10 min. The levels of phosphorylated Stat6 were detected using a maybe anti-pY641 Stat6 coupled to the fluorophore Alexi 488 (BD Bioscience). Analysis was performed on a Becton Dickinson LSRII equipped with 405, 488, and 640 nm lasers. Data analysis was performed in Citibank software. Log median fluorescence intensity values were plotted against cytokine concentration to yield dose-response curves.

TF-1 cells proliferation assay. TF-1 cells were seed to 2×10⁵ cells/ml in the presence of the indicated doses of IL-13 or the different IL-13 variants for 96 hr. Cells were washed 3× with cold (4C) PBS and fixed with 4% PFA for 15 min at room temperature. Number of cells in each well was determined by flow cytometry. Number of cells were represented as percentage and plotted against cytokine concentration to obtain dose-response curves.

Dendritic cells differentiation assay. CD14⁺ monocytes were isolated (>97% purity) from peripheral blood mononuclear cells by magnetic separation with anti-CD14 conjugated microbeads (Miltenyi Biotec). 5×10⁵ CD14⁺ monocytes were subsequently cultured with 50 ng/mL GM-CSF alone or with the indicated concentrations of IL-13 in the presence of 2 μg/ml of isotype control antibody, anti-IL-4Rα1 antibody or IL-13dn in 2 ml well plates containing IMDM medium (Gibco) supplemented with 10% human AB serum, 100 U/mL penicillin, 100 μg/mL streptomycin, 2 mM L-glutamine, sodium pyruvate, non-essential amino acids and 50 μM 2-ME. Cells were processed on day 6 with 5 mM EDTA and subsequently stained with DAPI (Invitrogen), fluorescently labeled isotype control mAbs, or mAbs against CD14, CD86, CD209 and HLA-DR (BD Biosciences). Dendritic cell differentiation was assessed by flow cytometry with a BD LSRII flow cytometer and median fluorescent intensities were generated by FlowJo (Treestar).

In vivo test of IL-13dn efficacy. 360 ng of mouse IL-13 were injected intra-tracheally with or without 150 μg of IL-13dn on days 0, 3 and 5. Lungs were harvest on day six. RNA was extracted and the expression levels of Muc5ac, Periostin, Arg1, CHIA, YM1, Fizz1were assessed by quantitative PCR.

All patents, patent publications, and other published references mentioned herein are hereby incorporated by reference in their entireties as if each had been individually and specifically incorporated by reference herein.

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined by reference to the appended claims, along with their full scope of equivalents.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. An immune cell targeting construct comprising:
an IL-13 superkine linked to the immune cell targeting construct, wherein the IL-13 superkine is engineered to have increased affinity for interleukin 13 receptor α2 (IL-13Rα2) relative to native human IL 13 protein; and decreased affinity for interleukin 13 receptor α1 (IL-13Rα1) relative to native human IL 13 protein, and wherein the IL-13 superkine comprises at least one amino acid change relative to the wild type IL-13 at one or more of positions selected from L10, R11, E12, I14, V18, R65, R86, D87, T88, K89, L101, K104, K105, F107, and R108.

2. The construct of claim 1 wherein the construct is a chimeric antigen receptor (CAR) and wherein the IL-13 superkine is fused to a transmembrane domain linked to an intracellular signaling region.

3. The construct of claim 2, wherein the intracellular signaling region comprises a CD3ζ signaling domain.

4. The construct of claim 2, wherein the intracellular signaling region comprises one or more of a CD28 signaling domain, a CD137 signaling domain, an OX-40 signaling domain, an ICOS signaling domain, a DAP10 signaling domain.

5. The construct of claim 1, wherein the construct is a T cell antigen coupler (TAC), wherein the IL-13 superkine is fused to a ligand that binds a protein associated with a T-cell receptor (TCR) complex; fused to a T cell receptor signaling domain polypeptide.

6. The construct of claim 5, wherein the protein associated with the TCR complex is CD3.

7. The construct of claim 5, wherein the T cell receptor signaling domain polypeptide comprises a CD4 cytosolic domain and a CD4 transmembrane domain.

8. The construct of claim 1, wherein the construct is an antibody coupled T cell receptors (ACTR), comprising a chimeric antigen receptor (CAR) component that binds to the IL-13 superkine at a high affinity.

9. The construct of claim 8, wherein the CAR component comprises CD16, and wherein the IL-13 superkine is fused to an Fc sequence.

10. The construct of claim 1, wherein the construct is a bispecific T cell engager (BiTE), wherein the IL-13 superkine is fused to a variable region of an antibody that binds to a component of a T cell receptor.

11. The construct of claim 10, wherein the component of a T cell receptor is CD3.

12. The construct of claim 1, wherein the IL-13 superkine comprises a set of amino acid substitutions selected from: [L10D, R11I, V18I, R86K, D87K, K89R, R108K]; [L10A, R86T, D87G, T88K, K89R, L101N, K104R, K105A, R108K]; [L10V, K89R, L101N, K105E, R108T]; [R11S, I14M, T88S, L101N, K105A, R108K]; [L10H, R11L, V18I, R86K, D87E, K89R, L101N, K105T, R108K]; [L10H, R11L, V18I, R86M, K89R, R108K]; [L10H, R86T, D87G, T88R, R108K]; [L10H, R86M, T88S, K89R, L101N, K104R, K105A, R108K]; and [L10A, V18F, R86K, K89R, L101I, K104R, R108K].

13. The construct of claim 12, wherein the IL-13 superkine comprises the set of amino acid substitutions: [L10H, R86T, D87G, T88R, R108K].

14. The construct according to claim 1, comprising an amino acid sequence set forth in SEQ ID NO:18 or SEQ ID NO:35, or comprising an amino acid sequence set forth in any of SEQ ID NO:2 through SEQ ID NO:38.

15. A nucleic acid encoding a construct of claim 1.

16. A vector comprising the nucleic acid of claim 15.

17. A T cell comprising a construct according to claim 1.

18. An NK cell comprising a construct according to claim 1.

19. The T cell of claim 17, wherein the T cell is a CD4+ T cell.

20. The T cell of claim 17, wherein the T cell is a CD8+ T cell.

21. An isolated population of immune cells of claim 17.

22. A pharmaceutical formulation comprising the immune cell population of claim 21.

23. A method of targeting a cell expressing an IL-13Rα2 receptor, the method comprising contacting the cell with a formulation of claim 22.

24. The method of claim 23, wherein the contacting is in vitro.

25. The method of claim 23, wherein the contacting is in vivo.

26. A method of treating cancer, the method comprising contacting an individual having cancer with an effective dose of the formulation of claim 22.

27. The method of claim 26, wherein the cancer is a leukemia, lymphoma, glioblastoma, medulloblastoma, breast cancer, head and neck cancer, kidney cancer, ovarian cancer, Kaposi's sarcoma, acute myelogenous leukemia, B-lineage malignancies, colorectal, pancreatic, kidney, or mesothelioma.

28. An isolated population of immune cells of claim 18.

29. A pharmaceutical formulation comprising the immune cell population of claim 28.

30. A method of targeting a cell expressing an IL-13Rα2 receptor, the method comprising contacting the cell with a formulation of claim 29.

31. The method of claim 30, wherein the contacting is in vitro.

32. The method of claim 30, wherein the contacting is in vivo.

33. A method of treating cancer, the method comprising contacting an individual having cancer with an effective dose of the formulation of claim 29.

34. The method of claim 33, wherein the cancer is a leukemia, lymphoma, glioblastoma, medulloblastoma, breast cancer, head and neck cancer, kidney cancer, ovarian cancer, Kaposi's sarcoma, acute myelogenous leukemia, B-lineage malignancies, colorectal, pancreatic, kidney, or mesothelioma.
